SEQUENCE LISTING (80 sequences)

SEQ ID NO: 1 - protein, 530 aa, synthetic construct
  SIGNAL 1..21: note = secretory leader
  CHAIN 24..129: note = anti-CD3 variable light chain
  CHAIN 130..144: note = linker
  CHAIN 145..263: note = anti-CD3 variable heavy chain
  CHAIN 264..273: note = linker
  CHAIN 279..284: note = 6x His Tag
  CHAIN 285..520: note = hinge
  CHAIN 521..530: note = HIV-Tat domain
MALLLCFVLLCGVVDFARSLSASDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSEQKLISEEDLNMHTGHHHHHHPRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGKGRKKRRQRRR

SEQ ID NO: 2 - protein, 566 aa, synthetic construct
  SIGNAL 1..21: note = secretory leader
  CHAIN 24..129: note = anti-CD3 variable light chain
  CHAIN 130..144: note = Gly-Ser linker
  CHAIN 145..263: note = anti-CD3 variable heavy chain
  CHAIN 264..273: note = linker
  CHAIN 279..284: note = 6x His Tag
  CHAIN 285..520: note = hinge
  CHAIN 521..550: note = fibritin domain
  CHAIN 557..566: note = HIV-Tat domain
  CHAIN 551..556: note = spacer
MALLLCFVLLCGVVDFARSLSASDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSEQKLISEEDLNMHTGHHHHHHPRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGKGYIPEAPRDGQAYVRKDGEWVLLSTFLSPAGGGGSGGRKKRRQRRR

SEQ ID NO: 3 - protein, 530 aa, synthetic construct
  SIGNAL 1..21: note = secretory leader
  CHAIN 24..142: note = anti-CD3 variable heavy chain
  CHAIN 143..157: note = Gly-Ser linker
  CHAIN 158..263: note = anti-CD3 variable light chain
  CHAIN 264..273: note = linker
  CHAIN 279..283: note = 6x His tag
  CHAIN 285..520: note = hinge
  CHAIN 521..530: note = HIV-Tat domain
MALLLCFVLLCGVVDFARSLSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKEQKLISEEDLNMHTGHHHHHHPRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGKGRKKRRQRRR

SEQ ID NO: 4 - protein, 566 aa, synthetic construct
  SIGNAL 1..21: note = secretory leader
  CHAIN 24..142: note = anti-CD3 variable heavy chain
  CHAIN 143..157: note = Gly-Ser linker
  CHAIN 158..263: note = anti-CD3 variable light chain
  CHAIN 264..273: note = linker
  CHAIN 279..284: note = 6x His Tag
  CHAIN 285..520: note = hinge
  CHAIN 521..560: note = fibritin domain
  CHAIN 557..566: note = HIV-Tat domain
  CHAIN 551..556: note = spacer
MALLLCFVLLCGVVDFARSLSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKEQKLISEEDLNMHTGHHHHHHPRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGKGYIPEAPRDGQAYVRKDGEWVLLSTFLSPAGGGGSGGRKKRRQRRR

SEQ ID NO: 5 - DNA, 1593 bp, synthetic construct
  source 1..1593: mol_type = other DNA
  CDS 1..69: protein_id = 14; translation = MALLLCFVLLCGVVDFARSLSAS
  CDS 70..387: note = anti-CD3 variable light chain; protein_id = 15; translation = DIQLTQSPAIMSASPGEKVTMTCRASSSVSYMNWYQQKSGTSPK RWIYDTSKVASGVPYRFSGSGSGTSYSLTISSMEAEDAATYYCQQWSSNPLTFGAGTK LELK
  CDS 388..432: note = GlySer linker; protein_id = 16; translation = GGGGSGGGGSGGGGS
  CDS 433..789: note = anti-CD3 variable heavy chain; protein_id = 17; translation = DIKLQQSGAELARPGASVKMSCKTSGYTFTRYTMHWVKQRPGQG LEWIGYINPSRGYTNYNQKFKDKATLTTDKSSSTAYMQLSSLTSEDSAVYYCARYYDD HYCLDYWGQGTTLTVSS
  CDS 790..852: note = mycHis tag; protein_id = 18; translation = EQKLISEEDLNMHTGHHHHHH
  CDS 853..1560: note = hinge; protein_id = 19; translation = PRGPTIKPCPPCKECHKCPAPNLEGGPSVFIFPPNIKDVLMISL TPKVTCVVVDVSEDDPDVRISWFVNNVEVHTAQTQTHREDYNSTIRVVSALPIQHQDW MSGKEFKCKVNNKDLPSPIERTISKIKGLVRAPQVYILPPPAEQLSRKDVSLTCLVVG FNPGDISVEWTSNGHTEENYKDTAPVLDSDGSYFIYSKLDIKTSKWEKTDSFSCNVRH EGLKNYYLKKTISRTPGK
  CDS 1561..1590: note = HIV-Tat domain; protein_id = 20; translation = GRKKRRQRRR
  CDS 1..1590: note = secretory leader peptide - human anti-CD3-scFv - Tag - hinge - HIV-Tat; protein_id = 61; translation = MALLLCFVLLCGVVDFARSLSASDIQLTQSPAIMSASPGEKVTM TCRASSSVSYMNWYQQKSGTSPKRWIYDTSKVASGVPYRFSGSGSGTSYSLTISSMEA EDAATYYCQQWSSNPLTFGAGTKLELKGGGGSGGGGSGGGGSDIKLQQSGAELARPGA SVKMSCKTSGYTFTRYTMHWVKQRPGQGLEWIGYINPSRGYTNYNQKFKDKATLTTDK SSSTAYMQLSSLTSEDSAVYYCARYYDDHYCLDYWGQGTTLTVSSEQKLISEEDLNMH TGHHHHHHPRGPTIKPCPPCKECHKCPAPNLEGGPSVFIFPPNIKDVLMISLTPKVTC VVVDVSEDDPDVRISWFVNNVEVHTAQTQTHREDYNSTIRVVSALPIQHQDWMSGKEF KCKVNNKDLPSPIERTISKIKGLVRAPQVYILPPPAEQLSRKDVSLTCLVVGFNPGDI SVEWTSNGHTEENYKDTAPVLDSDGSYFIYSKLDIKTSKWEKTDSFSCNVRHEGLKNY YLKKTISRTPGKGRKKRRQRRR
atggccctcctgctgtgcttcgtgctcctgtgcggagtggtggatttcgccagaagtttgagtgctagcgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaaggtggtggtggttctggcggcggcggctccggtggtggtggttctgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggcaggaagaagcggagacagcgacgaagatag

SEQ ID NO: 6 - DNA, 1701 bp, synthetic construct
  source 1..1701: mol_type = other DNA
  CDS 1..69: note = secretory leader; protein_id = 21; translation = MALLLCFVLLCGVVDFARSLSAS
  CDS 70..387: note = anti-CD3 variable light chain; protein_id = 22; translation = DIQLTQSPAIMSASPGEKVTMTCRASSSVSYMNWYQQKSGTSPK RWIYDTSKVASGVPYRFSGSGSGTSYSLTISSMEAEDAATYYCQQWSSNPLTFGAGTK LELK
  CDS 388..432: note = linker; protein_id = 23; translation = GGGGSGGGGSGGGGS
  CDS 433..789: note = anti-CD3 variable heavy chain; protein_id = 24; translation = DIKLQQSGAELARPGASVKMSCKTSGYTFTRYTMHWVKQRPGQG LEWIGYINPSRGYTNYNQKFKDKATLTTDKSSSTAYMQLSSLTSEDSAVYYCARYYDD HYCLDYWGQGTTLTVSS
  CDS 790..852: note = MycHis-Tag; protein_id = 25; translation = EQKLISEEDLNMHTGHHHHHH
  CDS 853..1650: note = Hinge; protein_id = 26; translation = PRGPTIKPCPPCKECHKCPAPNLEGGPSVFIFPPNIKDVLMISL TPKVTCVVVDVSEDDPDVRISWFVNNVEVHTAQTQTHREDYNSTIRVVSALPIQHQDW MSGKEFKCKVNNKDLPSPIERTISKIKGLVRAPQVYILPPPAEQLSRKDVSLTCLVVG FNPGDISVEWTSNGHTEENYKDTAPVLDSDGSYFIYSKLDIKTSKWEKTDSFSCNVRH EGLKNYYLKKTISRTPGKGYIPEAPRDGQAYVRKDGEWVLLSTFLSPA
  CDS 1669..1698: note = HIV Tat; protein_id = 27; translation = GRKKRRQRRR
  CDS 1651..1668: note = hydrophilic spacer; protein_id = 28; translation = GGGGSG
  CDS 1..1698: note = secretory leader peptide - human anti-CD3-scFv - Tag - hinge - fibritin - spacer - HIV-Tat; protein_id = 62; translation = MALLLCFVLLCGVVDFARSLSASDIQLTQSPAIMSASPGEKVTM TCRASSSVSYMNWYQQKSGTSPKRWIYDTSKVASGVPYRFSGSGSGTSYSLTISSMEA EDAATYYCQQWSSNPLTFGAGTKLELKGGGGSGGGGSGGGGSDIKLQQSGAELARPGA SVKMSCKTSGYTFTRYTMHWVKQRPGQGLEWIGYINPSRGYTNYNQKFKDKATLTTDK SSSTAYMQLSSLTSEDSAVYYCARYYDDHYCLDYWGQGTTLTVSSEQKLISEEDLNMH TGHHHHHHPRGPTIKPCPPCKECHKCPAPNLEGGPSVFIFPPNIKDVLMISLTPKVTC VVVDVSEDDPDVRISWFVNNVEVHTAQTQTHREDYNSTIRVVSALPIQHQDWMSGKEF KCKVNNKDLPSPIERTISKIKGLVRAPQVYILPPPAEQLSRKDVSLTCLVVGFNPGDI SVEWTSNGHTEENYKDTAPVLDSDGSYFIYSKLDIKTSKWEKTDSFSCNVRHEGLKNY YLKKTISRTPGKGYIPEAPRDGQAYVRKDGEWVLLSTFLSPAGGGGSGGRKKRRQRRR
atggccctcctgctgtgcttcgtgctcctgtgcggagtggtggatttcgccagaagtttgagtgctagcgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaaggtggtggtggttctggcggcggcggctccggtggtggtggttctgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggttatattcctgaagctccaagagatgggcaagcttacgttcgtaaagatggcgaatgggtattactttctacctttttatcaccagcaggaggaggagggtccggaggcaggaagaagcggagacagcgacgaagatag

SEQ ID NO: 7 - DNA, 1593 bp, synthetic construct
  source 1..1593: mol_type = other DNA
  CDS 1..69: note = secretory leader; protein_id = 29; translation = MALLLCFVLLCGVVDFARSLSAS
  CDS 70..423: note = anti-CD3 variable heavy chain; protein_id = 30; translation = DIKLQQSGAELARPGASVKMSCKTSGYTFTRYTMHWVKQRPGQG LEWIGYINPSRGYTNYNQKFKDKATLTTDKSSSTAYMQLSSLTSEDSAVYYCARYYDD HYCLDYWGQGTTLTVS
  CDS 424..471: note = linker; protein_id = 31; translation = SGGGGSGGGGSGGGGS
  CDS 472..789: note = anti-CD3 variable light chain; protein_id = 32; translation = DIQLTQSPAIMSASPGEKVTMTCRASSSVSYMNWYQQKSGTSPK RWIYDTSKVASGVPYRFSGSGSGTSYSLTISSMEAEDAATYYCQQWSSNPLTFGAGTK LELK
  CDS 790..852: note = MycHis-Tag; protein_id = 33; translation = EQKLISEEDLNMHTGHHHHHH
  CDS 853..1560: note = hinge; protein_id = 34; translation = PRGPTIKPCPPCKECHKCPAPNLEGGPSVFIFPPNIKDVLMISL TPKVTCVVVDVSEDDPDVRISWFVNNVEVHTAQTQTHREDYNSTIRVVSALPIQHQDW MSGKEFKCKVNNKDLPSPIERTISKIKGLVRAPQVYILPPPAEQLSRKDVSLTCLVVG FNPGDISVEWTSNGHTEENYKDTAPVLDSDGSYFIYSKLDIKTSKWEKTDSFSCNVRH EGLKNYYLKKTISRTPGK
  CDS 1561..1590: note = HIV-Tat; protein_id = 35; translation = GRKKRRQRRR
  CDS 1..1590: note = secretory leader peptide - human anti-CD3-scFv - Tag - hinge - HIV-Tat; protein_id = 63; translation = MALLLCFVLLCGVVDFARSLSASDIKLQQSGAELARPGASVKMS CKTSGYTFTRYTMHWVKQRPGQGLEWIGYINPSRGYTNYNQKFKDKATLTTDKSSSTA YMQLSSLTSEDSAVYYCARYYDDHYCLDYWGQGTTLTVSSGGGGSGGGGSGGGGSDIQ LTQSPAIMSASPGEKVTMTCRASSSVSYMNWYQQKSGTSPKRWIYDTSKVASGVPYRF SGSGSGTSYSLTISSMEAEDAATYYCQQWSSNPLTFGAGTKLELKEQKLISEEDLNMH TGHHHHHHPRGPTIKPCPPCKECHKCPAPNLEGGPSVFIFPPNIKDVLMISLTPKVTC VVVDVSEDDPDVRISWFVNNVEVHTAQTQTHREDYNSTIRVVSALPIQHQDWMSGKEF KCKVNNKDLPSPIERTISKIKGLVRAPQVYILPPPAEQLSRKDVSLTCLVVGFNPGDI SVEWTSNGHTEENYKDTAPVLDSDGSYFIYSKLDIKTSKWEKTDSFSCNVRHEGLKNY YLKKTISRTPGKGRKKRRQRRR
atggccctcctgctgtgcttcgtgctcctgtgcggagtggtggatttcgccagaagtttgagtgctagcgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggaggaggagggtctggagggggcggctccggagggggtggttctgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggcaggaagaagcggagacagcgacgaagatag

SEQ ID NO: 8 - DNA, 1701 bp, synthetic construct
  source 1..1701: mol_type = other DNA
  CDS 1..69: note = secretory leader; protein_id = 36; translation = MALLLCFVLLCGVVDFARSLSAS
  CDS 70..426: note = anti-CD3 variable heavy chain; protein_id = 37; translation = DIKLQQSGAELARPGASVKMSCKTSGYTFTRYTMHWVKQRPGQG LEWIGYINPSRGYTNYNQKFKDKATLTTDKSSSTAYMQLSSLTSEDSAVYYCARYYDD HYCLDYWGQGTTLTVSS
  CDS 427..471: note = Gly-Ser linker; protein_id = 38; translation = GGGGSGGGGSGGGGS
  CDS 472..789: note = anti-CD3 variable light chain; protein_id = 39; translation = DIQLTQSPAIMSASPGEKVTMTCRASSSVSYMNWYQQKSGTSPK RWIYDTSKVASGVPYRFSGSGSGTSYSLTISSMEAEDAATYYCQQWSSNPLTFGAGTK LELK
  CDS 790..852: note = MycHis Tag; protein_id = 40; translation = EQKLISEEDLNMHTGHHHHHH
  CDS 853..1560: note = hinge; protein_id = 41; translation = PRGPTIKPCPPCKECHKCPAPNLEGGPSVFIFPPNIKDVLMISL TPKVTCVVVDVSEDDPDVRISWFVNNVEVHTAQTQTHREDYNSTIRVVSALPIQHQDW MSGKEFKCKVNNKDLPSPIERTISKIKGLVRAPQVYILPPPAEQLSRKDVSLTCLVVG FNPGDISVEWTSNGHTEENYKDTAPVLDSDGSYFIYSKLDIKTSKWEKTDSFSCNVRH EGLKNYYLKKTISRTPGK
  CDS 1561..1659: note = fibritin; protein_id = 42; translation = GYIPEAPRDGQAYVRKDGEWVLLSTFLSPAGGG
  CDS 1651..1668: note = hydrophilic spacer; protein_id = 43; translation = GGGGSG
  CDS 1669..1698: note = HIV-Tat; protein_id = 44; translation = GRKKRRQRRR
  CDS 1..1698: note = secretory leader peptide - human anti-CD3-scFv - Tag - hinge - fibritin - spacer - HIV-Tat; protein_id = 64; translation = MALLLCFVLLCGVVDFARSLSASDIKLQQSGAELARPGASVKMS CKTSGYTFTRYTMHWVKQRPGQGLEWIGYINPSRGYTNYNQKFKDKATLTTDKSSSTA YMQLSSLTSEDSAVYYCARYYDDHYCLDYWGQGTTLTVSSGGGGSGGGGSGGGGSDIQ LTQSPAIMSASPGEKVTMTCRASSSVSYMNWYQQKSGTSPKRWIYDTSKVASGVPYRF SGSGSGTSYSLTISSMEAEDAATYYCQQWSSNPLTFGAGTKLELKEQKLISEEDLNMH TGHHHHHHPRGPTIKPCPPCKECHKCPAPNLEGGPSVFIFPPNIKDVLMISLTPKVTC VVVDVSEDDPDVRISWFVNNVEVHTAQTQTHREDYNSTIRVVSALPIQHQDWMSGKEF KCKVNNKDLPSPIERTISKIKGLVRAPQVYILPPPAEQLSRKDVSLTCLVVGFNPGDI SVEWTSNGHTEENYKDTAPVLDSDGSYFIYSKLDIKTSKWEKTDSFSCNVRHEGLKNY YLKKTISRTPGKGYIPEAPRDGQAYVRKDGEWVLLSTFLSPAGGGGSGGRKKRRQRRR
atggccctcctgctgtgcttcgtgctcctgtgcggagtggtggatttcgccagaagtttgagtgctagcgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggaggaggagggtctggagggggcggctccggagggggtggttctgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggttatattcctgaagctccaagagatgggcaagcttacgttcgtaaagatggcgaatgggtattactttctacctttttatcaccagcaggaggaggagggtccggaggcaggaagaagcggagacagcgacgaagatag

SEQ ID NO: 9 - protein, 526 aa, synthetic construct
  SIGNAL 1..21: note = secretory leader
  CHAIN 22..128: note = anti-CD3 variable light chain
  CA_BIND 129..143: note = Gly-Ser linker
  CHAIN 144..259: note = anti-CD3 variable heavy chain
  CHAIN 260..269: note = Myc Tag
  CHAIN 275..280: note = 6x His Tag
  CHAIN 281..516: note = hinge
  CHAIN 517..526: note = HIV-Tat domain
MALLLCFVLLCGVVDFARSLSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSEQKLISEEDLNMHTGHHHHHHPRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGKGRKKRRQRRR

SEQ ID NO: 10 - protein, 562 aa, synthetic construct
  SIGNAL 1..21: note = secretory leader
  CHAIN 22..128: note = anti-CD3 variable light chain
  CHAIN 129..143: note = Gly-Ser linker
  CHAIN 144..259: note = anti-CD3 variable heavy chain
  CHAIN 260..269: note = Myc Tag
  CHAIN 275..280: note = 6x His Tag
  CHAIN 281..516: note = hinge
  CHAIN 517..552: note = fibritin domain
  CHAIN 553..562: note = HIV Tat domain
MALLLCFVLLCGVVDFARSLSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSEQKLISEEDLNMHTGHHHHHHPRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGKGYIPEAPRDGQAYVRKDGEWVLLSTFLSPAGGGGSGGRKKRRQRRR

SEQ ID NO: 11 - DNA, 1581 bp, synthetic construct
  source 1..1581: mol_type = other DNA
  CDS 1..63: note = secretory leader; protein_id = 45; translation = MALLLCFVLLCGVVDFARSLS
  CDS 64..384: note = anti-CD3 variable light chain; protein_id = 46; translation = DIQMTQSPSSLPASLGDRVTINCQASQDISNYLNWYQQKPGKAP KLLIYYTNKLADGVPSRFSGSGSGRDSSFTISSLESEDIGSYYCQQYYNYPWTFGPGT KLEIK
  CDS 385..429: note = linker; protein_id = 47; translation = GGGGSGGGGSGGGGS
  CDS 430..777: note = anti-CD3 variable heavy chain; protein_id = 48; translation = EVQLVESGGGLVQPGKSLKLSCEASGFTFSGYGMHWVRQAPGRG LESVAYITSSSINIKYADAVKGRFTVSRDNAKNLLFLQMNILKSEDTAMYYCARFDWD KNYWGQGTMVTVSS
  CDS 778..840: note = MycHis Tag; protein_id = 49; translation = EQKLISEEDLNMHTGHHHHHH
  CDS 841..1548: note = hinge; protein_id = 50; translation = PRGPTIKPCPPCKECHKCPAPNLEGGPSVFIFPPNIKDVLMISL TPKVTCVVVDVSEDDPDVRISWFVNNVEVHTAQTQTHREDYNSTIRVVSALPIQHQDW MSGKEFKCKVNNKDLPSPIERTISKIKGLVRAPQVYILPPPAEQLSRKDVSLTCLVVG FNPGDISVEWTSNGHTEENYKDTAPVLDSDGSYFIYSKLDIKTSKWEKTDSFSCNVRH EGLKNYYLKKTISRTPGK
  CDS 1549..1578: note = HIV-Tat; protein_id = 51; translation = GRKKRRQRRR
  CDS 1..1578: note = secretory leader peptide - murine anti-CD3-scFv -Tag - hinge - HIV-Tat; protein_id = 65; translation = MALLLCFVLLCGVVDFARSLSDIQMTQSPSSLPASLGDRVTINC QASQDISNYLNWYQQKPGKAPKLLIYYTNKLADGVPSRFSGSGSGRDSSFTISSLESE DIGSYYCQQYYNYPWTFGPGTKLEIKGGGGSGGGGSGGGGSEVQLVESGGGLVQPGKS LKLSCEASGFTFSGYGMHWVRQAPGRGLESVAYITSSSINIKYADAVKGRFTVSRDNA KNLLFLQMNILKSEDTAMYYCARFDWDKNYWGQGTMVTVSSEQKLISEEDLNMHTGHH HHHHPRGPTIKPCPPCKECHKCPAPNLEGGPSVFIFPPNIKDVLMISLTPKVTCVVVD VSEDDPDVRISWFVNNVEVHTAQTQTHREDYNSTIRVVSALPIQHQDWMSGKEFKCKV NNKDLPSPIERTISKIKGLVRAPQVYILPPPAEQLSRKDVSLTCLVVGFNPGDISVEW TSNGHTEENYKDTAPVLDSDGSYFIYSKLDIKTSKWEKTDSFSCNVRHEGLKNYYLKK TISRTPGKGRKKRRQRRR
atggccctcctgctgtgcttcgtgctcctgtgcggagtggtggatttcgccagaagtttgagtgacatccagatgacccagtctccatcatcactgcctgcctccctgggagacagagtcactatcaattgtcaggccagtcaggacattagcaattatttgaactggtaccagcagaaaccagggaaagctcctaagctcctgatctattatacaaataaattggcagatggagtcccatcaaggttcagtggcagtggctctgggagagattcttctttcactatcagcagcctggaatccgaagatattggatcttattactgtcagcagtattataactatccctggaccttcggacctggcaccaagctggaaatcaaaggaggaggagggtccggagggggcggatctggagggggaggctctgaggtgcagctggtggagtctgggggaggcttggtgcagcctggaaagtccctgaaactctcctgtgaggcctctggattcaccttcagcggctatggcatgcactgggtccgccaggctccagggagggggctggagtccgtcgcatacattactagtagtagtattaatatcaaatatgctgacgctgtgaaaggccggttcaccgtctccagagacaatgccaagaacttgctgtttctgcagatgaacattctcaagtctgaggacacagccatgtactactgtgcaagattcgactgggacaaaaattactggggccagggaaccatggtcaccgtctcctcagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggcaggaagaagcggagacagcgacgaagatag

SEQ ID NO: 12 - DNA, 1689 bp, synthetic construct
  source 1..1689: mol_type = other DNA
  CDS 1..63: note = secretory leader; protein_id = 52; translation = MALLLCFVLLCGVVDFARSLS
  CDS 64..384: note = anti-CD3 variable light chain; protein_id = 53; translation = DIQMTQSPSSLPASLGDRVTINCQASQDISNYLNWYQQKPGKAP KLLIYYTNKLADGVPSRFSGSGSGRDSSFTISSLESEDIGSYYCQQYYNYPWTFGPGT KLEIK
  CDS 385..429: note = linker; protein_id = 54; translation = GGGGSGGGGSGGGGS
  CDS 430..777: note = anti-CD3 variable heavy chain; protein_id = 55; translation = EVQLVESGGGLVQPGKSLKLSCEASGFTFSGYGMHWVRQAPGRG LESVAYITSSSINIKYADAVKGRFTVSRDNAKNLLFLQMNILKSEDTAMYYCARFDWD KNYWGQGTMVTVSS
  CDS 778..840: note = MycHis Tag; protein_id = 56; translation = EQKLISEEDLNMHTGHHHHHH
  CDS 841..1548: note = hinge; protein_id = 57; translation = PRGPTIKPCPPCKECHKCPAPNLEGGPSVFIFPPNIKDVLMISL TPKVTCVVVDVSEDDPDVRISWFVNNVEVHTAQTQTHREDYNSTIRVVSALPIQHQDW MSGKEFKCKVNNKDLPSPIERTISKIKGLVRAPQVYILPPPAEQLSRKDVSLTCLVVG FNPGDISVEWTSNGHTEENYKDTAPVLDSDGSYFIYSKLDIKTSKWEKTDSFSCNVRH EGLKNYYLKKTISRTPGK
  CDS 1549..1638: note = fibritin domain; protein_id = 58; translation = GYIPEAPRDGQAYVRKDGEWVLLSTFLSPA
  CDS 1639..1656: note = hydrophilic spacer; protein_id = 59; translation = GGGGSG
  CDS 1657..1686: note = HIV Tat domain; protein_id = 60; translation = GRKKRRQRRR
  CDS 1..1686: note = secretory leader peptide - human anti-CD3-scFv - Tag - hinge - fibritin - spacer - HIV-Tat; protein_id = 66; translation = MALLLCFVLLCGVVDFARSLSDIQMTQSPSSLPASLGDRVTINC QASQDISNYLNWYQQKPGKAPKLLIYYTNKLADGVPSRFSGSGSGRDSSFTISSLESE DIGSYYCQQYYNYPWTFGPGTKLEIKGGGGSGGGGSGGGGSEVQLVESGGGLVQPGKS LKLSCEASGFTFSGYGMHWVRQAPGRGLESVAYITSSSINIKYADAVKGRFTVSRDNA KNLLFLQMNILKSEDTAMYYCARFDWDKNYWGQGTMVTVSSEQKLISEEDLNMHTGHH HHHHPRGPTIKPCPPCKECHKCPAPNLEGGPSVFIFPPNIKDVLMISLTPKVTCVVVD VSEDDPDVRISWFVNNVEVHTAQTQTHREDYNSTIRVVSALPIQHQDWMSGKEFKCKV NNKDLPSPIERTISKIKGLVRAPQVYILPPPAEQLSRKDVSLTCLVVGFNPGDISVEW TSNGHTEENYKDTAPVLDSDGSYFIYSKLDIKTSKWEKTDSFSCNVRHEGLKNYYLKK TISRTPGKGYIPEAPRDGQAYVRKDGEWVLLSTFLSPAGGGGSGGRKKRRQRRR
atggccctcctgctgtgcttcgtgctcctgtgcggagtggtggatttcgccagaagtttgagtgacatccagatgacccagtctccatcatcactgcctgcctccctgggagacagagtcactatcaattgtcaggccagtcaggacattagcaattatttgaactggtaccagcagaaaccagggaaagctcctaagctcctgatctattatacaaataaattggcagatggagtcccatcaaggttcagtggcagtggctctgggagagattcttctttcactatcagcagcctggaatccgaagatattggatcttattactgtcagcagtattataactatccctggaccttcggacctggcaccaagctggaaatcaaaggaggaggagggtccggagggggcggatctggagggggaggctctgaggtgcagctggtggagtctgggggaggcttggtgcagcctggaaagtccctgaaactctcctgtgaggcctctggattcaccttcagcggctatggcatgcactgggtccgccaggctccagggagggggctggagtccgtcgcatacattactagtagtagtattaatatcaaatatgctgacgctgtgaaaggccggttcaccgtctccagagacaatgccaagaacttgctgtttctgcagatgaacattctcaagtctgaggacacagccatgtactactgtgcaagattcgactgggacaaaaattactggggccagggaaccatggtcaccgtctcctcagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggttatattcctgaagctccaagagatgggcaagcttacgttcgtaaagatggcgaatgggtattactttctacctttttatcaccagcaggaggaggagggtccggaggcaggaagaagcggagacagcgacgaagatag

SEQ ID NO: 13 - DNA, 37135 bp, synthetic construct
  source 1..37135: mol_type = other DNA
  5'UTR 1..103: function = L-ITR
  3'UTR 37099..37135: function = R-ITR
  regulatory 380..625: regulatory_class = promoter
  gene 727..1646: gene = delta AA 4-25 variant of E1A gene
  misc_feature 837..857: note = RNAi target site
  misc_feature 886..909: note = RNAi target site
  misc_feature 1647..1764: note = heterologous target sites for RNAi inserted into the 3-UTR of E1A, equivalent to E1A-886-909, E1A-837-857, Prot IX-7467-7487
  regulatory 1813..1910: regulatory_class = promoter
  CDS 1911..2441: note = E1B-19k; protein_id = 67; translation = MEAWECLEDFSAVRNLLEQSSNSTSWFWRFLWGSSQAKLVCRIK EDYKWEFEELLKSCGELFDSLNLGHQALFQEKVIKTLDFSTPGRAAAAVAFLSFIKDK WSEETHLSGGYLLDFLAMHLWRAVVRHKNRLLLLSSVRPAIIPTEEQQQQQEEARRRR QEQSPWNPRAGLDPRE
  CDS 2216..3706: note = E1B-55k; protein_id = 68; translation = MERRNPSERGVPAGFSGHASVESGCETQESPATVVFRPPGDNTD GGAAAAAGGSQAAAAGAEPMEPESRPGPSGMNVVQVAELYPELRRILTITEDGQGLKG VKRERGACEATEEARNLAFSLMTRHRPECITFQQIKDNCANELDLLAQKYSIEQLTTY WLQPGDDFEEAIRVYAKVALRPDCKYKISKLVNIRNCCYISGNGAEVEIDTEDRVAFR CSMINMWPGVLGMDGVVIMNVRFTGPNFSGTVFLANTNLILHGVSFYGFNNTCVEAWT DVRVRGCAFYCCWKGVVCRPKSRASIKKCLFERCTLGILSEGNSRVRHNVASDCGCFM LVKSVAVIKHNMVCGNCEDRASQMLTCSDGNCHLLKTIHVASHSRKAWPVFEHNILTR CSLHLGNRRGVFLPYQCNLSHTKILLEPESMSKVNLNGVFDMTMKIWKVLRYDETRTR CRPCECGGKHIRNQPVMLDVTEELRPDHLVLACTRAEFGSSDEDTD
  regulatory 3737..4320: regulatory_class = ribosome_binding_site
  CDS 4339..5919: note = fusion protein; protein_id = 69; translation = MALLLCFVLLCGVVDFARSLSDIQMTQSPSSLPASLGDRVTINC QASQDISNYLNWYQQKPGKAPKLLIYYTNKLADGVPSRFSGSGSGRDSSFTISSLESE DIGSYYCQQYYNYPWTFGPGTKLEIKGGGGSGGGGSGGGGSEVQLVESGGGLVQPGKS LKLSCEASGFTFSGYGMHWVRQAPGRGLESVAYITSSSINIKYADAVKGRFTVSRDNA KNLLFLQMNILKSEDTAMYYCARFDWDKNYWGQGTMVTVSSEQKLISEEDLNMHTGHH HHHHPRGPTIKPCPPCKECHKCPAPNLEGGPSVFIFPPNIKDVLMISLTPKVTCVVVD VSEDDPDVRISWFVNNVEVHTAQTQTHREDYNSTIRVVSALPIQHQDWMSGKEFKCKV NNKDLPSPIERTISKIKGLVRAPQVYILPPPAEQLSRKDVSLTCLVVGFNPGDISVEW TSNGHTEENYKDTAPVLDSDGSYFIYSKLDIKTSKWEKTDSFSCNVRHEGLKNYYLKK TISRTPGKGRKKRRQRRR
  regulatory 5986..6344: note = prMinRGC; regulatory_class = promoter
  misc_feature 6439..6501: note = inhibitory shRNA for E1A
  misc_feature 6373..6435: note = inhibitory shRNA for E1A (1)
  misc_feature 6625..6681: note = inhibitory shRNA for Prot IX
  misc_feature 6748..6804: note = inhibitory shRNA for E4 orf6 (1)
  misc_feature 6936..6996: note = inhibitory shRNA for E4 orf6
  misc_feature 6996..7053: note = inhibitory shRNA for TP
  misc_feature 7120..7178: note = inhibitory shRNA for AdPol
  gene 7446..7868: note = protein IX, part of viral backbone
  gene 9034..17956: note = polymerase, Ad-Pol, part of viral backbone, reverse complementary
  gene 12420..17956: gene = terminal protein (TP), part of viral backbone, reverse complementary
  CDS 22677..25535: note = hexon, part of backbone; protein_id = 78; translation = MATPSMMPQWSYMHISGQDASEYLSPGLVQFARATETYFSLNNK FRNPTVAPTHDVTTDRSQRLTLRFIPVDREDTAYSYKARFTLAVGDNRVLDMASTYFD IRGVLDRGPTFKPYSGTAYNALAPKGAPNPCEWDEAATALEINLEEEDDDNEDEVDEQ AEQQKTHVFGQAPYSGINITKEGIQIGVEGQTPKYADKTFQPEPQIGESQWYETEINH AAGRVLKKTTPMKPCYGSYAKPTNENGGQGILVKQQNGKLESQVEMQFFSTTEAAAGN GDNLTPKVVLYSEDVDIETPDTHISYMPTIKEGNSRELMGQQSMPNRPNYIAFRDNFI GLMYYNSTGNMGVLAGQASQLNAVVDLQDRNTELSYQLLLDSIGDRTRYFSMWNQAVD SYDPDVRIIENHGTEDELPNYCFPLGGVINTETLTKVKPKTGQENGWEKDATEFSDKN EIRVGNNFAMEINLNANLWRNFLYSNIALYLPDKLKYSPSNVKISDNPNTYDYMNKRV VAPGLVDCYINLGARWSLDYMDNVNPFNHHRNAGLRYRSMLLGNGRYVPFHIQVPQKF FAIKNLLLLPGSYTYEWNFRKDVNMVLQSSLGNDLRVDGASIKFDSICLYATFFPMAH NTASTLEAMLRNDTNDQSFNDYLSAANMLYPIPANATNVPISIPSRNWAAFRGWAFTR LKTKETPSLGSGYDPYYTYSGSIPYLDGTFYLNHTFKKVAITFDSSVSWPGNDRLLTP NEFEIKRSVDGEGYNVAQCNMTKDWFLVQMLANYNIGYQGFYIPESYKDRMYSFFRNF QPMSRQVVDDTKYKDYQQVGILHQHNNSGFVGYLAPTMREGQAYPANFPYPLIGKTAV DSITQKKFLCDRTLWRIPFSSNFMSMGALTDLGQNLLYANSAHALDMTFEVDPMDEPT LLYVLFEVFDVVRVHQPHRGVIETVYLRTPFSAGNATT
  gene 26280..27869: gene = reverse complementary sequence of DNA-binding protein (dbd), part of viral backbone
  misc_feature 31703: note = delta E3, equivalent to deletion of nt 28137..30818 of wildtype virus
  CDS 32222..33967: note = fiber, part of viral backbone; protein_id = 70; translation = MKRARPSEDTFNPVYPYDTETGPPTVPFLTPPFVSPNGFQESPP GVLSLRLSEPLVTSNGMLALKMGNGLSLDEAGNLTSQNVTTVSPPLKKTKSNINLEIS APLTVTSEALTVAAAAPLMVAGNTLTMQSQAPLTVHDSKLSIATQGPLTVSEGKLALQ TSGPLTTTDSSTLTITASPPLTTATGSLGIDLKEPIYTQNGKLGLKYGAPLHVTDDLN TLTVATGPGVTINNTSLQTKVTGALGFDSQGNMQLNVAGGLRIDSQNRRLILDVSYPF DAQNQLNLRLGQGPLFINSAHNLDINYNKGLYLFTASNNSKKLEVNLSTAKGLMFDAT AIAINAGDGLEFGSPNAPNTNPLKTKIGHGLEFDSNKAMVPKLGTGLSFDSTGAITVG NKNNDKLTLWTTPAPSPNCRLNAEKDAKLTLVLTKCGSQILATVSVLAVKGSLAPISG TVQSAHLIIRFDENGVLLNNSFLDPEYWNFRNGDLTEGTAYTNAVGFMPNLSAYPKSH GKTAKSNIVSQVYLNGDKTKPVTLTITLNGTQETGDTTPSAYSMSFSWDWSGHNYINE IFATSSYTFSYIAQE
  gene 34094..35257: gene = reverse complementary sequence of E4 orf6/7
  gene 35178..35492: gene = reverse complementary sequence of hybrid E4 orf4, Ad5/Ad11
  regulatory 35861..35863: regulatory_class = terminator
  misc_feature 35861..35884: note = inactivated E4 orf3 locus, reverse complementary
  gene 35881..36291: note = E4 orf2, reverse complementary
  gene 36321..36707: note = E4 orf1 reverse complementary
  misc_feature 37099..37135: note = LTR, r-ITR
  gene 27896..30319: gene = 100k, part of viral backbone
  misc_feature 6501..6558: note = inhibitory shRNA for E1A (2)
  misc_feature 6870..6930: note = inhibitory shRNA for E4 orf6 (2)
  CDS 17992..19707: note = Prot III: penton, part of backbone; protein_id = 77; translation = MRRAAMYEEGPPPSYESVVSAAPVAAALGSPFDAPLDPPFVPPR YLRPTGGRNSIRYSELAPLFDTTRVYLVDNKSTDVASLNYQNDHSNFLTTVIQNNDYS PGEASTQTINLDDRSHWGGDLKTILHTNMPNVNEFMFTNKFKARVMVSRLPTKDNQVE LKYEWVEFTLPEGNYSETMTIDLMNNAIVEHYLKVGRQNGVLESDIGVKFDTRNFRLG FDPVTGLVMPGVYTNEAFHPDIILLPGCGVDFTHSRLSNLLGIRKRQPFQEGFRITYD DLEGGNIPALLDVDAYQASLKDDTEQGGGGAGGSNSSGSGAEENSNAAAAAMQPVEDM NDHAIRGDTFATRAEEKRAEAEAAAEAAAPAAQPEVEKPQKKPVIKPLTEDSKKRSYN LISNDSTFTQYRSWYLAYNYGDPQTGIRSWTLLCTPDVTCGSEQVYWSLPDMMQDPVT FRSTRQISNFPVVGAELLPVHSKSFYNDQAVYSQLIRQFTSLTHVFNRFPENQILARP PAPTITTVSENVPALTDHGTLPLRNSIGGVQRVTITDARRRTCPYVYKALGIVSPRVL SSRTF
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgtaactataacggtcctaaggtagcgaaagctagtaccatggtgatgcggttttggcagtacatcaatggcggtggatagcggtccgactcacggggatttccaagtctccaccccattgacgtcaatgggagtttgttttggcaccaaaatcaacgggactttccaaaatgtcgtaacaactccgccccacggacgcaaatgggccgtaggcgtgtacggtgggcggtctatataagcccggctggtttagtgaaccgtcagatcctcgagggcgtgtagtgtatttatacccggtgagttcctcaagaggccactcttgagtgccagcgagtagagttttctcctccgagccgctccgacaccgggactgaaaatgagacatgaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacacacaggaggcggtttcgcagatttttacacacagaagggattgacttactcacttttacacacaggaagcattgtgagctcatatgagacccaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgatcacgcgtttcgaacgatttatggccctcctgctgtgcttcgtgctcctgtgcggagtggtggatttcgccagaagtttgagtgacatccagatgacccagtctccatcatcactgcctgcctccctgggagacagagtcactatcaattgtcaggccagtcaggacattagcaattatttgaactggtaccagcagaaaccagggaaagctcctaagctcctgatctattatacaaataaattggcagatggagtcccatcaaggttcagtggcagtggctctgggagagattcttctttcactatcagcagcctggaatccgaagatattggatcttattactgtcagcagtattataactatccctggaccttcggacctggcaccaagctggaaatcaaaggaggaggagggtccggagggggcggatctggagggggaggctctgaggtgcagctggtggagtctgggggaggcttggtgcagcctggaaagtccctgaaactctcctgtgaggcctctggattcaccttcagcggctatggcatgcactgggtccgccaggctccagggagggggctggagtccgtcgcatacattactagtagtagtattaatatcaaatatgctgacgctgtgaaaggccggttcaccgtctccagagacaatgccaagaacttgctgtttctgcagatgaacattctcaagtctgaggacacagccatgtactactgtgcaagattcgactgggacaaaaattactggggccagggaaccatggtcaccgtctcctcagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggcaggaagaagcggagacagcgacgaagatagaaataaaggtaccttattttcattagatttgtgtgttggttttttgtgtggcggcctcgagatgggagagcttgatgccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcagggccaggcaagtccaggcaggccaggcaagtccaggcaggccaggcaagtccaggcaggatcgaattcgagctcgtacggtgggagggctatataagcagacctcgtttagtgaaccgtcagatcgcctggagacgaagcttgctagcggatcctcgagaaggtatattgctgttgacagtgagcgagaagggattgacttactcacttttgtgaagccacagatgaaaagtgagtaagtcaatcccttcgtgcctactgcctcggaattcactagcggatcctcgagaaggtatattgctgttgacagtgagcgaggaggcggtttcgcagattttgtgaagccacagatgaaaatctgcgaaaccgcctccgtgcctactgcctcggaattcactagcggatcctcgagaaggtatattgctgttgacagtgagcgaggaagcattgtgagctcatatgtgaagccacagatgatatgagctcacaatgcttccgtgcctactgcctcggaattcactagcggatcctcgagaaggtatattgctgttgacagtgagcgagacaggaaaccgtgtggaatagtgaagccacagatgtattccacacggtttcctgtcgtgcctactgcctcggaattcactagcggatcctcgaaaggtatattgctgttgacagtgagcgaccatgtttaatcagaggtttatagtgaagccacagatgtataaacctctgattaaacatgggtgcctactgcctcggaattcactagcggatcctcgagaaggtatattgctgttgacagtgagcgacggagtacctacacaacaattgtgaagccacagatgaattgttgtgtaggtactccggtgcctactgcctcggaattcactagcggatcctcgagaaggtatattgctgttgacagtgagcgagcgaatacgtgcagctaaacatgtgaagccacagatgatgtttagctgcacgtattcgcgtgcctactgcctcggaattcactagctagtcatatgctgcagtgtagatctaaataaagcaatagcatcacaaataaagcatttcattcactgcattctagttgtggtttgtacgcgtgatatcaattcatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaagattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtcctgccgcttagcgtgttccgtgtgatagttcaagtacaaccacactcttaagttggtcaaaagaatgctggcttcagttgtaatcaaaactccatcgcatctaatcgttctgaggaaatcatccacggtagcatatgcaaatcccaaccaagcaatgcaactggattgtgtttcaagcaggagaggagagggaagagacggaagaaccatgttaatttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttacagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttacatcgttaattaa

SEQ ID NO: 14 - protein, 23 aa, synthetic construct
MALLLCFVLLCGVVDFARSLSAS

SEQ ID NO: 15 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 16 - protein, 15 aa, synthetic construct
GGGGSGGGGSGGGGS

SEQ ID NO: 17 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 18 - protein, 21 aa, synthetic construct
EQKLISEEDLNMHTGHHHHHH

SEQ ID NO: 19 - protein, 236 aa, synthetic construct
PRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGK

SEQ ID NO: 20 - protein, 10 aa, synthetic construct
GRKKRRQRRR

SEQ ID NO: 21 - protein, 23 aa, synthetic construct
MALLLCFVLLCGVVDFARSLSAS

SEQ ID NO: 22 - protein, 106 aa, synthetic construct; identical to SEQ ID NO: 15

SEQ ID NO: 23 - protein, 15 aa, synthetic construct
GGGGSGGGGSGGGGS

SEQ ID NO: 24 - protein, 119 aa, synthetic construct; identical to SEQ ID NO: 17

SEQ ID NO: 25 - protein, 21 aa, synthetic construct
EQKLISEEDLNMHTGHHHHHH

SEQ ID NO: 26 - protein, 266 aa, synthetic construct
PRGPTIKPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGKGYIPEAPRDGQAYVRKDGEWVLLSTFLSPA

SEQ ID NO: 27 - protein, 10 aa, synthetic construct
GRKKRRQRRR

SEQ ID NO: 28 - protein, 6 aa, synthetic construct
GGGGSG

SEQ ID NO: 29 - protein, 23 aa, synthetic construct
MALLLCFVLLCGVVDFARSLSAS

SEQ ID NO: 30 - protein, 118 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVS

SEQ ID NO: 31 - protein, 16 aa, synthetic construct
SGGGGSGGGGSGGGGS

SEQ ID NO: 32 - protein, 106 aa, synthetic construct; identical to SEQ ID NO: 15

SEQ ID NO: 33 - protein, 21 aa, synthetic construct
EQKLISEEDLNMHTGHHHHHH

SEQ ID NO: 34 - protein, 236 aa, synthetic construct; identical to SEQ ID NO: 19

SEQ ID NO: 35 - protein, 10 aa, synthetic construct
GRKKRRQRRR

SEQ ID NO: 36 - protein, 23 aa, synthetic construct
MALLLCFVLLCGVVDFARSLSAS

SEQ ID NO: 37 - protein, 119 aa, synthetic construct; identical to SEQ ID NO: 17

SEQ ID NO: 38 - protein, 15 aa, synthetic construct
GGGGSGGGGSGGGGS

SEQ ID NO: 39 - protein, 106 aa, synthetic construct; identical to SEQ ID NO: 15

SEQ ID NO: 40 - protein, 21 aa, synthetic construct
EQKLISEEDLNMHTGHHHHHH

SEQ ID NO: 41 - protein, 236 aa, synthetic construct; identical to SEQ ID NO: 19

SEQ ID NO: 42 - protein, 33 aa, synthetic construct
GYIPEAPRDGQAYVRKDGEWVLLSTFLSPAGGG

SEQ ID NO: 43 - protein, 6 aa, synthetic construct
GGGGSG

SEQ ID NO: 44 - protein, 10 aa, synthetic construct
GRKKRRQRRR

SEQ ID NO: 45 - protein, 21 aa, synthetic construct
MALLLCFVLLCGVVDFARSLS

SEQ ID NO: 46 - protein, 107 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 47 - protein, 15 aa, synthetic construct
GGGGSGGGGSGGGGS

SEQ ID NO: 48 - protein, 116 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 49 - protein, 21 aa, synthetic construct
EQKLISEEDLNMHTGHHHHHH

SEQ ID NO: 50 - protein, 236 aa, synthetic construct; identical to SEQ ID NO: 19

SEQ ID NO: 51 - protein, 10 aa, synthetic construct
GRKKRRQRRR

SEQ ID NO: 52 - protein, 21 aa, synthetic construct
MALLLCFVLLCGVVDFARSLS

SEQ ID NO: 53 - protein, 107 aa, synthetic construct; identical to SEQ ID NO: 46

SEQ ID NO: 54 - protein, 15 aa, synthetic construct
GGGGSGGGGSGGGGS

SEQ ID NO: 55 - protein, 116 aa, synthetic construct; identical to SEQ ID NO: 48

SEQ ID NO: 56 - protein, 21 aa, synthetic construct
EQKLISEEDLNMHTGHHHHHH

SEQ ID NO: 57 - protein, 236 aa, synthetic construct; identical to SEQ ID NO: 19

SEQ ID NO: 58 - protein, 30 aa, synthetic construct
GYIPEAPRDGQAYVRKDGEWVLLSTFLSPA

SEQ ID NO: 59 - protein, 6 aa, synthetic construct
GGGGSG

SEQ ID NO: 60 - protein, 10 aa, synthetic construct
GRKKRRQRRR

SEQ ID NO: 61 - protein, 530 aa, synthetic construct; identical to SEQ ID NO: 1

SEQ ID NO: 62 - protein, 566 aa, synthetic construct; identical to SEQ ID NO: 2

SEQ ID NO: 63 - protein, 530 aa, synthetic construct; identical to SEQ ID NO: 3

SEQ ID NO: 64 - protein, 566 aa, synthetic construct; identical to SEQ ID NO: 4

SEQ ID NO: 65 - protein, 526 aa, synthetic construct; identical to SEQ ID NO: 9

SEQ ID NO: 66 - protein, 562 aa, synthetic construct; identical to SEQ ID NO: 10

SEQ ID NO: 67 - protein, 176 aa, synthetic construct
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQQQEEARRRRQEQSPWNPRAGLDPRE

SEQ ID NO: 68 - protein, 496 aa, synthetic construct
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 69 - protein, 526 aa, synthetic construct; identical to SEQ ID NO: 9

SEQ ID NO: 70 - protein, 581 aa, synthetic construct
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 71 - DNA, 37243 bp, synthetic construct
  source 1..37243: mol_type = other DNA
  5'UTR 1..103: function = L-ITR
  3'UTR 37141..37243: function = R-ITR
  regulatory 380..625: note = CMVs; regulatory_class = promoter
  gene 727..1646: note = reverse complementary sequence for delta AA 4-25 variant of E1A gene
  misc_feature 837..857: note = RNAi target site
  misc_feature 886..909: note = RNAi target site
  misc_feature 1647..1764: note = heterologous target sites for RNAi inserted into the 3-UTR of E1A, equivalent to E1A-886-909, E1A-837-857, Prot IX-7467-7487
  regulatory 1813..1910: note = promoter for E1B; regulatory_class = promoter
  CDS 1911..2441: note = E1B-19k; protein_id = 72; translation = MEAWECLEDFSAVRNLLEQSSNSTSWFWRFLWGSSQAKLVCRIK EDYKWEFEELLKSCGELFDSLNLGHQALFQEKVIKTLDFSTPGRAAAAVAFLSFIKDK WSEETHLSGGYLLDFLAMHLWRAVVRHKNRLLLLSSVRPAIIPTEEQQQQQEEARRRR QEQSPWNPRAGLDPRE
  CDS 2216..3706: note = E1B-55k; protein_id = 73; translation = MERRNPSERGVPAGFSGHASVESGCETQESPATVVFRPPGDNTD GGAAAAAGGSQAAAAGAEPMEPESRPGPSGMNVVQVAELYPELRRILTITEDGQGLKG VKRERGACEATEEARNLAFSLMTRHRPECITFQQIKDNCANELDLLAQKYSIEQLTTY WLQPGDDFEEAIRVYAKVALRPDCKYKISKLVNIRNCCYISGNGAEVEIDTEDRVAFR CSMINMWPGVLGMDGVVIMNVRFTGPNFSGTVFLANTNLILHGVSFYGFNNTCVEAWT DVRVRGCAFYCCWKGVVCRPKSRASIKKCLFERCTLGILSEGNSRVRHNVASDCGCFM LVKSVAVIKHNMVCGNCEDRASQMLTCSDGNCHLLKTIHVASHSRKAWPVFEHNILTR CSLHLGNRRGVFLPYQCNLSHTKILLEPESMSKVNLNGVFDMTMKIWKVLRYDETRTR CRPCECGGKHIRNQPVMLDVTEELRPDHLVLACTRAEFGSSDEDTD
  regulatory 3737..4320: note = IRES; regulatory_class = ribosome_binding_site
  CDS 4339..6027: note = fusion protein; protein_id = 74; translation = MALLLCFVLLCGVVDFARSLSDIQMTQSPSSLPASLGDRVTINC QASQDISNYLNWYQQKPGKAPKLLIYYTNKLADGVPSRFSGSGSGRDSSFTISSLESE DIGSYYCQQYYNYPWTFGPGTKLEIKGGGGSGGGGSGGGGSEVQLVESGGGLVQPGKS LKLSCEASGFTFSGYGMHWVRQAPGRGLESVAYITSSSINIKYADAVKGRFTVSRDNA KNLLFLQMNILKSEDTAMYYCARFDWDKNYWGQGTMVTVSSEQKLISEEDLNMHTGHH HHHHPRGPTIKPCPPCKECHKCPAPNLEGGPSVFIFPPNIKDVLMISLTPKVTCVVVD VSEDDPDVRISWFVNNVEVHTAQTQTHREDYNSTIRVVSALPIQHQDWMSGKEFKCKV NNKDLPSPIERTISKIKGLVRAPQVYILPPPAEQLSRKDVSLTCLVVGFNPGDISVEW TSNGHTEENYKDTAPVLDSDGSYFIYSKLDIKTSKWEKTDSFSCNVRHEGLKNYYLKK TISRTPGKGYIPEAPRDGQAYVRKDGEWVLLSTFLSPAGGGGSGGRKKRRQRRR
  regulatory 6094..6452: note = prMinRGC; regulatory_class = promoter
  misc_feature 6481..6543: note = inhibitory shRNA for E1A (1)
  misc_feature 6609..6666: note = inhibitory shRNA for E1A (2)
  misc_feature 6733..6789: note = inhibitory shRNA for Prot IX
  misc_feature 6856..6912: note = inhibitory shRNA for E4orf6 (1)
  misc_feature 6978..7038: note = inhibitory shRNA for E4orf6 (2)
  misc_feature 7104..7161: note = inhibitory shRNA for TP
  misc_feature 7228..7286: note = inhibitory shRNA for AdPol
  gene 9142..18064: gene = reverse complementary for polymerase Ad-Pol, part of backbone
  gene 7554..7976: note = protein IX, part of backbone
  gene 12528..18064: note = reverse complementary for terminal protein TP, part of backbone
  CDS 18100..19815: note = Prot III, penton, part of backbone; protein_id = 79; translation = MRRAAMYEEGPPPSYESVVSAAPVAAALGSPFDAPLDPPFVPPR YLRPTGGRNSIRYSELAPLFDTTRVYLVDNKSTDVASLNYQNDHSNFLTTVIQNNDYS PGEASTQTINLDDRSHWGGDLKTILHTNMPNVNEFMFTNKFKARVMVSRLPTKDNQVE LKYEWVEFTLPEGNYSETMTIDLMNNAIVEHYLKVGRQNGVLESDIGVKFDTRNFRLG FDPVTGLVMPGVYTNEAFHPDIILLPGCGVDFTHSRLSNLLGIRKRQPFQEGFRITYD DLEGGNIPALLDVDAYQASLKDDTEQGGGGAGGSNSSGSGAEENSNAAAAAMQPVEDM NDHAIRGDTFATRAEEKRAEAEAAAEAAAPAAQPEVEKPQKKPVIKPLTEDSKKRSYN LISNDSTFTQYRSWYLAYNYGDPQTGIRSWTLLCTPDVTCGSEQVYWSLPDMMQDPVT FRSTRQISNFPVVGAELLPVHSKSFYNDQAVYSQLIRQFTSLTHVFNRFPENQILARP PAPTITTVSENVPALTDHGTLPLRNSIGGVQRVTITDARRRTCPYVYKALGIVSPRVL SSRTF
  CDS 22785..25643: note = hexon, part of backbone; protein_id = 80; translation = MATPSMMPQWSYMHISGQDASEYLSPGLVQFARATETYFSLNNK FRNPTVAPTHDVTTDRSQRLTLRFIPVDREDTAYSYKARFTLAVGDNRVLDMASTYFD IRGVLDRGPTFKPYSGTAYNALAPKGAPNPCEWDEAATALEINLEEEDDDNEDEVDEQ AEQQKTHVFGQAPYSGINITKEGIQIGVEGQTPKYADKTFQPEPQIGESQWYETEINH AAGRVLKKTTPMKPCYGSYAKPTNENGGQGILVKQQNGKLESQVEMQFFSTTEAAAGN GDNLTPKVVLYSEDVDIETPDTHISYMPTIKEGNSRELMGQQSMPNRPNYIAFRDNFI GLMYYNSTGNMGVLAGQASQLNAVVDLQDRNTELSYQLLLDSIGDRTRYFSMWNQAVD SYDPDVRIIENHGTEDELPNYCFPLGGVINTETLTKVKPKTGQENGWEKDATEFSDKN EIRVGNNFAMEINLNANLWRNFLYSNIALYLPDKLKYSPSNVKISDNPNTYDYMNKRV VAPGLVDCYINLGARWSLDYMDNVNPFNHHRNAGLRYRSMLLGNGRYVPFHIQVPQKF FAIKNLLLLPGSYTYEWNFRKDVNMVLQSSLGNDLRVDGASIKFDSICLYATFFPMAH NTASTLEAMLRNDTNDQSFNDYLSAANMLYPIPANATNVPISIPSRNWAAFRGWAFTR LKTKETPSLGSGYDPYYTYSGSIPYLDGTFYLNHTFKKVAITFDSSVSWPGNDRLLTP NEFEIKRSVDGEGYNVAQCNMTKDWFLVQMLANYNIGYQGFYIPESYKDRMYSFFRNF QPMSRQVVDDTKYKDYQQVGILHQHNNSGFVGYLAPTMREGQAYPANFPYPLIGKTAV DSITQKKFLCDRTLWRIPFSSNFMSMGALTDLGQNLLYANSAHALDMTFEVDPMDEPT LLYVLFEVFDVVRVHQPHRGVIETVYLRTPFSAGNATT
  gene 26388..27977: note = reverse complementary for DNA-binding protein dbp, part of backbone
  CDS 28004..30427: note = 100k, part of backbone; protein_id = 75; translation = MESVEKKDSLTAPSEFATTASTDAANAPTTFPVEAPPLEEEEVI IEQDPGFVSEDDEDRSVPTEDKKQDQDNAEANEEQVGRGDERHGDYLDVGDDVLLKHL QRQCAIICDALQERSDVPLAIADVSLAYERHLFSPRVPPKRQENGTCEPNPRLNFYPV FAVPEVLATYHIFFQNCKIPLSCRANRSRADKQLALRQGAVIPDIASLNEVPKIFEGL GRDEKRAANALQQENSENESHSGVLVELEGDNARLAVLKRSIEVTHFAYPALNLPPKV MSTVMSELIVRRAQPLERDANLQEQTEEGLPAVGDEQLARWLQTREPADLEERRKLMM AAVLVTVELECMQRFFADPEMQRKLEETLHYTFRQGYVRQACKISNVELCNLVSYLGI LHENRLGQNVLHSTLKGEARRDYVRDCVYLFLCYTWQTAMGVWQQCLEECNLKELQKL LKQNLKDLWTAFNERSVAAHLADIIFPERLLKTLQQGLPDFTSQSMLQNFRNFILERS GILPATCCALPSDFVPIKYRECPPPLWGHCYLLQLANYLAYHSDIMEDVSGDGLLECH CRCNLCTPHRSLVCNSQLLNESQIIGTFELQGPSPDEKSAAPGLKLTPGLWTSAYLRK FVPEDYHAHEIRFYEDQSRPPNAELTACVITQGHILGQLQAINKARQEFLLRKGRGVY LDPQSGEELNPIPPPPQPYQQQPRALASQDGTQKEAAAAAATHGRGGILGQSGRGGFG RGGGGHDGRLGEPRRGSFRGRRGVRRNTVTLGRIPLAGAPEIGNRFQHGYNLRSSGAA GTARSPTQP
  misc_feature 31811: note = delta E3, equivalent to deletion of 28137-30818 of the wild-type virus
  CDS 32330..34075: note = fiber, part of backbone; protein_id = 76; translation = MKRARPSEDTFNPVYPYDTETGPPTVPFLTPPFVSPNGFQESPP GVLSLRLSEPLVTSNGMLALKMGNGLSLDEAGNLTSQNVTTVSPPLKKTKSNINLEIS APLTVTSEALTVAAAAPLMVAGNTLTMQSQAPLTVHDSKLSIATQGPLTVSEGKLALQ TSGPLTTTDSSTLTITASPPLTTATGSLGIDLKEPIYTQNGKLGLKYGAPLHVTDDLN TLTVATGPGVTINNTSLQTKVTGALGFDSQGNMQLNVAGGLRIDSQNRRLILDVSYPF DAQNQLNLRLGQGPLFINSAHNLDINYNKGLYLFTASNNSKKLEVNLSTAKGLMFDAT AIAINAGDGLEFGSPNAPNTNPLKTKIGHGLEFDSNKAMVPKLGTGLSFDSTGAITVG NKNNDKLTLWTTPAPSPNCRLNAEKDAKLTLVLTKCGSQILATVSVLAVKGSLAPISG TVQSAHLIIRFDENGVLLNNSFLDPEYWNFRNGDLTEGTAYTNAVGFMPNLSAYPKSH GKTAKSNIVSQVYLNGDKTKPVTLTITLNGTQETGDTTPSAYSMSFSWDWSGHNYINE IFATSSYTFSYIAQE
  gene 34202..35365: note = reverse complementary for E4orf6/7
  gene 35286..35600: note = reverse complementary for hybrid E4orf4, Ad5/Ad11
  regulatory 35969..35971: note = reverse complementary stop codon for E4orf3; regulatory_class = terminator
  misc_feature 35969..35992: note = reverse complementary for inactivated E4orf3 locus
  gene 35989..36399: note = reverse complementary for E4orf2
  gene 36429..36815: note = reverse complementary for E4orf1
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgtaactataacggtcctaaggtagcgaaagctagtaccatggtgatgcggttttggcagtacatcaatggcggtggatagcggtccgactcacggggatttccaagtctccaccccattgacgtcaatgggagtttgttttggcaccaaaatcaacgggactttccaaaatgtcgtaacaactccgccccacggacgcaaatgggccgtaggcgtgtacggtgggcggtctatataagcccggctggtttagtgaaccgtcagatcctcgagggcgtgtagtgtatttatacccggtgagttcctcaagaggccactcttgagtgccagcgagtagagttttctcctccgagccgctccgacaccgggactgaaaatgagacatgaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacacacaggaggcggtttcgcagatttttacacacagaagggattgacttactcacttttacacacaggaagcattgtgagctcatatgagacccaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgatcacgcgtttcgaacgatttatggccctcctgctgtgcttcgtgctcctgtgcggagtggtggatttcgccagaagtttgagtgacatccagatgacccagtctccatcatcactgcctgcctccctgggagacagagtcactatcaattgtcaggccagtcaggacattagcaattatttgaactggtaccagcagaaaccagggaaagctcctaagctcctgatctattatacaaataaattggcagatggagtcccatcaaggttcagtggcagtggctctgggagagattcttctttcactatcagcagcctggaatccgaagatattggatcttattactgtcagcagtattataactatccctggaccttcggacctggcaccaagctggaaatcaaaggaggaggagggtccggagggggcggatctggagggggaggctctgaggtgcagctggtggagtctgggggaggcttggtgcagcctggaaagtccctgaaactctcctgtgaggcctctggattcaccttcagcggctatggcatgcactgggtccgccaggctccagggagggggctggagtccgtcgcatacattactagtagtagtattaatatcaaatatgctgacgctgtgaaaggccggttcaccgtctccagagacaatgccaagaacttgctgtttctgcagatgaacattctcaagtctgaggacacagccatgtactactgtgcaagattcgactgggacaaaaattactggggccagggaaccatggtcaccgtctcctcagaacagaaactcatctcagaagaggatctgaatatgcataccggacatcatcaccatcaccatcccagagggcccacaatcaagccctgtcctccatgcaaggagtgtcacaaatgcccagctcctaacctcgagggcggaccatccgtcttcatcttccctccaaatatcaaggatgtgctcatgatctccctgacacccaaggtcacctgtgtggtggtggatgtgagcgaggatgacccagacgtccggatcagctggtttgtgaacaacgtggaagtgcacacagctcagacacagacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctggtcagagctccacaggtgtacatcttgcctccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggctcttacttcatctacagcaagctcgatatcaaaacaagcaagtgggagaaaacagattccttctcatgcaacgtgagacacgagggcctgaaaaattactacctgaagaagaccatctcccggactcctggcaaaggttatattcctgaagctccaagagatgggcaagcttacgttcgtaaagatggcgaatgggtattactttctacctttttatcaccagcaggaggaggagggtccggaggcaggaagaagcggagacagcgacgaagatagaaataaaggtaccttattttcattagatttgtgtgttggttttttgtgtggcggcctcgagatgggagagcttgatgccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcagggccaggcaagtccaggcaggccaggcaagtccaggcaggccaggcaagtccaggcaggatcgaattcgagctcgtacggtgggagggctatataagcagacctcgtttagtgaaccgtcagatcgcctggagacgaagcttgctagcggatcctcgagaaggtatattgctgttgacagtgagcgagaagggattgacttactcacttttgtgaagccacagatgaaaagtgagtaagtcaatcccttcgtgcctactgcctcggaattcactagcggatcctcgagaaggtatattgctgttgacagtgagcgaggaggcggtttcgcagattttgtgaagccacagatgaaaatctgcgaaaccgcctccgtgcctactgcctcggaattcactagcggatcctcgagaaggtatattgctgttgacagtgagcgaggaagcattgtgagctcatatgtgaagccacagatgatatgagctcacaatgcttccgtgcctactgcctcggaattcactagcggatcctcgagaaggtatattgctgttgacagtgagcgagacaggaaaccgtgtggaatagtgaagccacagatgtattccacacggtttcctgtcgtgcctactgcctcggaattcactagcggatcctcgaaaggtatattgctgttgacagtgagcgaccatgtttaatcagaggtttatagtgaagccacagatgtataaacctctgattaaacatgggtgcctactgcctcggaattcactagcggatcctcgagaaggtatattgctgttgacagtgagcgacggagtacctacacaacaattgtgaagccacagatgaattgttgtgtaggtactccggtgcctactgcctcggaattcactagcggatcctcgagaaggtatattgctgttgacagtgagcgagcgaatacgtgcagctaaacatgtgaagccacagatgatgtttagctgcacgtattcgcgtgcctactgcctcggaattcactagctagtcatatgctgcagtgtagatctaaataaagcaatagcatcacaaataaagcatttcattcactgcattctagttgtggtttgtacgcgtgatatcaattcatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaagattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtcctgccgcttagcgtgttccgtgtgatagttcaagtacaaccacactcttaagttggtcaaaagaatgctggcttcagttgtaatcaaaactccatcgcatctaatcgttctgaggaaatcatccacggtagcatatgcaaatcccaaccaagcaatgcaactggattgtgtttcaagcaggagaggagagggaagagacggaagaaccatgttaatttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttacagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttacatcgttaattaa

SEQ ID NO: 72 - protein, 176 aa, synthetic construct; identical to SEQ ID NO: 67

SEQ ID NO: 73 - protein, 496 aa, synthetic construct; identical to SEQ ID NO: 68

SEQ ID NO: 74 - protein, 562 aa, synthetic construct; identical to SEQ ID NO: 10

SEQ ID NO: 75 - protein, 807 aa, synthetic construct
MESVEKKDSLTAPSEFATTASTDAANAPTTFPVEAPPLEEEEVIIEQDPGFVSEDDEDRSVPTEDKKQDQDNAEANEEQVGRGDERHGDYLDVGDDVLLKHLQRQCAIICDALQERSDVPLAIADVSLAYERHLFSPRVPPKRQENGTCEPNPRLNFYPVFAVPEVLATYHIFFQNCKIPLSCRANRSRADKQLALRQGAVIPDIASLNEVPKIFEGLGRDEKRAANALQQENSENESHSGVLVELEGDNARLAVLKRSIEVTHFAYPALNLPPKVMSTVMSELIVRRAQPLERDANLQEQTEEGLPAVGDEQLARWLQTREPADLEERRKLMMAAVLVTVELECMQRFFADPEMQRKLEETLHYTFRQGYVRQACKISNVELCNLVSYLGILHENRLGQNVLHSTLKGEARRDYVRDCVYLFLCYTWQTAMGVWQQCLEECNLKELQKLLKQNLKDLWTAFNERSVAAHLADIIFPERLLKTLQQGLPDFTSQSMLQNFRNFILERSGILPATCCALPSDFVPIKYRECPPPLWGHCYLLQLANYLAYHSDIMEDVSGDGLLECHCRCNLCTPHRSLVCNSQLLNESQIIGTFELQGPSPDEKSAAPGLKLTPGLWTSAYLRKFVPEDYHAHEIRFYEDQSRPPNAELTACVITQGHILGQLQAINKARQEFLLRKGRGVYLDPQSGEELNPIPPPPQPYQQQPRALASQDGTQKEAAAAAATHGRGGILGQSGRGGFGRGGGGHDGRLGEPRRGSFRGRRGVRRNTVTLGRIPLAGAPEIGNRFQHGYNLRSSGAAGTARSPTQP

SEQ ID NO: 76 - protein, 581 aa, synthetic construct; identical to SEQ ID NO: 70

SEQ ID NO: 77 - protein, 571 aa, synthetic construct
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 78 - protein, 952 aa, synthetic construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 79 - protein, 571 aa, synthetic construct; identical to SEQ ID NO: 77

SEQ ID NO: 80 - protein, 952 aa, synthetic construct; identical to SEQ ID NO: 78

(19) United States
(12) Patent Application Publication
(10) Pub. No.: US 2026/0242470 A1
(43) Pub. Date: Aug. 20, 2026

(54) FUSION PROTEIN FOR LOCALIZED ACTIVATION OF T-CELLS

(71) Applicant: Medizinische Hochschule Hannover, Hannover (DE)

(72) Inventors: Florian Kühnel, Hannover (DE); Thomas Wirth, Hannover (DE); Malin Peter, Hannover (DE)

(21) Appl. No.: 19/165,422

(22) PCT Filed: Mar. 15, 2024
(86) PCT No.: PCT/EP2024/056981

§ 371 Date: Sep. 15, 2025

(30) Foreign Application Priority Data
Mar. 17, 2023 (EP) 23162746.4

(51) Int. Cl. C07K 16/28; A61K 35/768; A61P 35/00; C07K 14/005

(52) U.S. Cl. CPC C07K 16/2809; A61K 35/768; A61P 35/00; C07K 14/005; C07K 16/2818; C07K 2317/622; C07K 2319/70

(57) ABSTRACT

The invention provides a fusion protein and a nucleic acid construct encoding the fusion protein, preferably an oncolytic viral particle that contains the nucleic acid construct encoding the fusion protein, wherein the fusion protein from N-terminus to C-terminus comprises or consists of a secretory leader peptide-anti-CD3-antibody section-hinge-HIV-Tat.

The present invention relates to a fusion protein for localized activation of cytotoxic T-cells, especially for use in the treatment of solid tumours, and to viral particles that contain a nucleic acid construct that encodes the fusion protein, which viral particles are for use in the treatment of solid tumours. Presence of the fusion protein in a tumour has the advantage of activating an immune response of cytotoxic T-cells against the tumour, especially T-cells specifically directed against a tumour antigen. The fusion protein of the invention, e.g. when expressed from a nucleic acid construct contained in a viral particle, has the advantage of activating T-cells at the location of expression of the fusion protein, especially when expressed within tumour tissue, with little or no activity to activate T-cells outside the tumour in which the fusion protein is expressed. The fusion protein of the invention has furthermore the advantage to bind to tissue, e.g. tumour tissue, and to activate T-cells at the site of its presence, independent of the presence of a particular tumour-specific antigen in the tissue. Accordingly, the fusion protein can be used in the treatment of tumours in the absence of a determination of tumour-specific antigens, and for use in the treatment of tumours that are devoid of a tumour-specific antigen.

Preferably, the fusion protein of the invention is for use in activating T-cells within a tissue, e.g. for use in the treatment of a tumour, wherein the fusion protein is present, e.g. expressed in or introduced into the tissue, e.g. tumour tissue, for activating T-cells in this tissue.

The fusion protein preferably is encoded by a nucleic acid construct contained in an oncolytic viral particle, such that administration of the viral particle to a human results in pre-dominant presence of the viral particle in the vicinity of or within tumour tissue and expression of the fusion protein in the vicinity of or within tumour tissue. Generally preferred, the fusion protein is encoded by a nucleic acid construct packaged in a viral particle for transient expression only. The viral particle preferably is an oncolytic virus.

### STATE OF THE ART

Liao et al., Cancer Gene Therapy (2003) 10, 779-790, for use in tumour treatment describe expression of fusion proteins containing an anti-CD3-scFv, a linker and a transmembrane domain in cells for activating T-cells against the cells expressing the fusion protein. The fusion protein was produced in cultivated cells by transient transfection using lipofectamine in vitro. For activating T-cells, this publication concludes that it is important to reduce shedding of such fusion protein from cells that express this fusion protein.

Liao et Roffler, Gene Therapy (2000) 7, 339-347, describe activation of cytotoxic T-cells by expressing a fusion protein of anti-CD3-scFv-hinge region of IgG-transmembrane domain and cytosolic domain of CD80 as a surface-bound protein after transfection into in vitro cultivated tumour cells.

WO2021/239586 A1 describes oncolytic viruses which have an inactivated E4 orf3 and a mutated E4 orf4.

Kulkarni et al., Biochemical and Biophysical Research Communications 70-712 (2005) claims extracellular expression of a fusion protein consisting of Tat-LAMP (CD107b, lysosomal associated membrane protein). The ability of surface-bound entire Tat to activate PBMC was tested by expression of the Tat-LAMP fusion on HEK293 cells, with increased activation of PBMC by added anti-CD3.

Lapidot and Litovchick, Drug Development Research 502-515 (2000) is an overview of HIV-Tat antagonists for therapy of AIDS.

Zauli et al., J Immunol 2216-2224 (1996) describes that anti-CD3 antibody and entire HIV-1 Tat when co-immobilized on plastic induce proliferation of CD4+ T-cells.

Chirmule et al., J Virol. 492-498 (1995) contacts LiCl-pretreated T-cells with entire Tat and then adds tetanus antigen-pulsed APC in order to analyze HIV infection pathways.

Lim et al., PLOS ONE (2013) describes endocytosis of a cell-penetrating peptide fused to an anti-Ras scFv and of a Tat-scFv fusion as a comparison.

Linkers for use in fusion proteins are described by Chen et al., Adv Drug Deliv. Rev. 1357-1369 (2013).

Urba et al., Cancer Res 2394-2401 (1992) relates to anti-CD3 antibody for use in tumor treatment.

US 2015/0098960 A1 describes a fusion protein comprising a transcription factor domain of Tat, a Cystein-rich domain and a C-terminal domain of Tat for treating cancer.

Wang et al., BMC Biotechnology (2017) 17:57 describes improving catalytic activity of lichenase and xylanase by a C-terminal fusion to a foldon domain, resulting in trimerization.

### OBJECT OF THE INVENTION

It is an object of the invention to provide an alternative, preferably an improved fusion protein for use in the treatment of tumours, especially for activating cytotoxic T-cells, and a carrier for the fusion protein for use in the treatment of tumours.

### DESCRIPTION OF THE INVENTION

The invention achieves the object by the features of the claims and especially by a fusion protein and a nucleic acid construct encoding the fusion protein, preferably an oncolytic viral particle that contains the nucleic acid construct encoding the fusion protein, wherein the fusion protein from N-terminus to C-terminus comprises or consists of a secretory leader peptide-anti-CD3-antibody section-hinge-HIV-Tat, with optionally a Tag-domain between the scFv and the hinge, wherein the anti-CD3-antibody section preferably is an anti-CD3-scFv domain, e.g. a single-chain antibody of a variable light chain (VL), a linker and a variable heavy chain (VH) VL-linker-VH or VH-linker-VL, which linker is e.g. a Gly-Ser-linker, wherein the Tag-domain is e.g. a mycHis-Tag that can be detected by an anti-Myc-antibody and that contains a His-oligomer for affinity purification, wherein the HIV-Tat is an electropositive section of the HIV-Tat protein from the HIV, and therefore herein the protein transduction domain of HIV-Tat is also designated as HIV-Tat domain, and in short HIV-Tat, as a preferred representative of a protein transduction domain. Generally herein, the anti-CD3-antibody section is represented by an anti-CD3 scFv domain.

It was found that the anti-CD3-scFv domain of the fusion protein results in activation of cytotoxic T-cells inside a solid tumour and/or results in invasion of active cytotoxic T-cells into a solid tumour, wherein the activation is not limited to those tumour cells that express the fusion protein, but the activation of cytotoxic T-cells also occurs in the tumour-tissue vicinity of those tumour cells that express the fusion protein. It is believed that the secretion of the fusion protein, as caused by its secretory leader peptide and lack of a transmembrane domain and lack of intracellular domains also results in spreading of the fusion protein within tumour tissue, whereas the HIV-Tat domain prevents an unlimited distribution of the fusion protein but results in a gradual diffusion of the fusion protein with preference into tissue within the direct vicinity of cells expressing the fusion protein. It is currently assumed that the HIV-Tat domain after secretion from expressing cells results in the fusion protein sticking to tissue, e.g. tumour tissue, and presence of the fusion protein activates cytotoxic T-cells. Therefore, the fusion protein has the advantage of activating cytotoxic T-cells also in tumour cells that are not transduced by viral particles to express the fusion protein, but which tumour cells are only in the vicinity of cells expressing the fusion protein. Because the cell-binding properties of the fusion protein containing the HIV-Tat domain are not dependent on presence of a specific antigen in the tissue, i.e. the cell-binding properties of the fusion protein are not restricted to a specific tumor antigen, expression of the fusion protein does not limit the broad range applicability of the carrier viral particle encoding the fusion protein. Accordingly, a viral particle, preferably an oncolytic virus, encoding the fusion protein is suitable for use in the activation of T-cells, e.g. for use in the treatment of a tumour, in the tissue present in the vicinity of the cells that are infected by the viral particle. Therein, the localization of the activation of T-cells is determined by the specificity of the viral particle, e.g. specificity for a target tissue, e.g. tumour tissue.

As the fusion protein is secreted from cells that are infected by a virus encoding the fusion protein, the activity of the fusion protein to activate T-cells, especially within tissue in the vicinity of the infected cells, is independent from the type of the virus.

A viral particle, preferably an oncolytic viral particle, that contains a nucleic acid construct encoding the fusion protein is used as a carrier, resulting in expression of the fusion protein by cells, preferably tumour cells, of the recipient human. As an alternative to a viral particle, liposomes can be used as a carrier, the liposomes containing a nucleic acid encoding the fusion protein. As an embodiment, a nucleic acid construct encoding the fusion protein is contained in liposomes, wherein the liposomes preferably contain a protein providing for tumour-specificity, e.g. the liposomes may have a liposome-membrane bound tumour-specific ligand, e.g. an antibody that is specific for a tumour-specific antigen, which antibody is bound to the liposomal membrane. Alternatively or additionally, the liposomes can be for use in direct intra-tumoural injection.

Generally herein, protein sequences and arrangements of domains of the fusion protein are described from N-terminus to C-terminus.

An exemplary fusion protein consisting of secretory leader peptide-anti-CD3-scFv-Tag-hinge-HIV-Tat is
MALLLCFVLLCGVVDFARSLSASDIQLTQSPAIMSASPGEKVTMTCRASSSVSYMNW YQQKSGTSPKRWIYDTSKVASGVPYRFSGSGSGTSYSLTISSMEAEDAATYYCQQWS SNPLTFGAGTKLELKGGGGSGGGGSGGGGSDIKLQQSGAELARPGASVKMSCKTSG YTFTRYTMHWVKQRPGQGLEWIGYINPSRGYTNYNQKFKDKATLTTDKSSSTAYMQ LSSLTSEDSAVYYCARYYDDHYCLDYWGQGTTLTVSSEQKLISEEDLNMHTGHHHH HHPRGPTIKPCPPCKECHKCPAPNLEGGPSVFIFPPNIKDVLMISLTPKVTCVVVDVSE DDPDVRISWFVNNVEVHTAQTQTHREDYNSTIRVVSALPIQHQDWMSGKEFKCKVN NKDLPSPIERTISKIKGLVRAPQVYILPPPAEQLSRKDVSLTCLVVGFNPGDISVEWTSN GHTEENYKDTAPVLDSDGSYFIYSKLDIKTSKWEKTDSFSCNVRHEGLKNYYLKKTIS RTPGKGRKKRRQRRR
(SEQ ID NO: 1), containing an anti-CD3 scFV as VL-hinge-VH, specific for the human CD3, with a 6× His tag as an optional Tag, so that the fusion protein may consist of amino acids No. 1 . . . 278 fused to amino acids 285 . . . 530 of SEQ ID NO: 1,
and
MALLLCFVLLCGVVDFARSLSASDIKLQQSGAELARPGASVKMSCKTSGYTFTRYTM HWVKQRPGQGLEWIGYINPSRGYTNYNQKFKDKATLTTDKSSSTAYMQLSSLTSEDS AVYYCARYYDDHYCLDYWGQGTTLTVSSGGGGSGGGGSGGGGSDIQLTQSPAIMSA SPGEKVTMTCRASSSVSYMNWYQQKSGTSPKRWIYDTSKVASGVPYRFSGSGSGTS YSLTISSMEAEDAATYYCQQWSSNPLTFGAGTKLELKEQKLISEEDLNMHTGHHHHH HPRGPTIKPCPPCKECHKCPAPNLEGGPSVFIFPPNIKDVLMISLTPKVTCVVVDVSED DPDVRISWFVNNVEVHTAQTQTHREDYNSTIRVVSALPIQHQDWMSGKEFKCKVNN KDLPSPIERTISKIKGLVRAPQVYILPPPAEQLSRKDVSLTCLVVGFNPGDISVEWTSNG HTEENYKDTAPVLDSDGSYFIYSKLDIKTSKWEKTDSFSCNVRHEGLKNYYLKKTISR TPGKGRKKRRQRRR
(SEQ ID NO: 3), containing the anti-CD3 scFV as VH-hinge-VL, specific for the human CD3, with a 6× His tag as an optional Tag, so that the fusion protein may consist of amino acids No. 1 . . . 273 fused to amino acids 285 . . . 566 of SEQ ID NO: 2. The fusion protein of the invention can have an amino acid sequence having a sequence identity or homology of at least 80%, of at least 85%, of at least 90%, of at least 95% to one of, or to two of the sequences SEQ ID NO: 1, and SEQ ID NO: 3, optionally with an additional oligomerizing domain, preferably in combination with a hydrophilic spacer, arranged between the hinge and the HIV-Tat, e.g. an amino acid sequence having a sequence identity or homology of at least 80%, of at least 85%, of at least 90%, of at least 95% to one of, or to two of the sequences SEQ ID NO: 2, and SEQ ID NO: 4.

In an embodiment, the fusion protein can consist of a secretory leader peptide-anti-CD3-antibody section-hinge-PTD, e.g. secretory leader peptide-anti-CD3-antibody section-hinge-HIV-Tat.

In a preferred embodiment, the fusion protein between its hinge and HIV-Tat contains an oligomerizing domain that results in e.g. dimerization, trimerization of secreted fusion proteins. A preferred oligomerizing domain is the fibritin domain. In this embodiment, the fusion protein from N-terminus to C-terminus comprises or consists of a secretory leader peptide-anti-CD3 antibody section-hinge-oligomerizing domain-HIV-Tat, with optionally a Tag-domain between the scFv and the hinge, wherein the anti-CD3 antibody section is an anti-CD3-scFv is a single-chain antibody of a variable light chain (VL), a linker and a variable heavy chain (VH) in the arrangement of VL-linker-VH or VH-linker-VL, which linker is e.g. a Gly-Ser-linker, wherein the Tag-domain is e.g. a MycHis-Tag that can be detected by an anti-Myc-antibody and that contains a His-oligomer for affinity purification by metal ion affinity, wherein the HIV-Tat is an electropositive section of the HIV-Tat domain from the HIV.

An exemplary fusion protein consisting of secretory leader peptide-anti-CD3-scFv-Tag-hinge-oligomerizing domain-HIV-Tat is
MALLLCFVLLCGVVDFARSLSASDIQLTQSPAIMSASPGEKVTMTCRASSSVSYMNW YQQKSGTSPKRWIYDTSKVASGVPYRFSGSGSGTSYSLTISSMEAEDAATYYCQQWS SNPLTFGAGTKLELKGGGGSGGGGSGGGGSDIKLQQSGAELARPGASVKMSCKTSG YTFTRYTMHWVKQRPGQGLEWIGYINPSRGYTNYNQKFKDKATLTTDKSSSTAYMQ LSSLTSEDSAVYYCARYYDDHYCLDYWGQGTTLTVSSEQKLISEEDLNMHTGHHHH HHPRGPTIKPCPPCKECHKCPAPNLEGGPSVFIFPPNIKDVLMISLTPKVTCVVVDVSE DDPDVRISWFVNNVEVHTAQTQTHREDYNSTIRVVSALPIQHQDWMSGKEFKCKVN NKDLPSPIERTISKIKGLVRAPQVYILPPPAEQLSRKDVSLTCLVVGFNPGDISVEWTSN GHTEENYKDTAPVLDSDGSYFIYSKLDIKTSKWEKTDSFSCNVRHEGLKNYYLKKTIS RTPGKGYIPEAPRDGQAYVRKDGEWVLLSTFLSPAGGGGSGGRKKRRQRRR (SEQ ID NO: 2), containing the anti-CD3 scFV as VH-hinge-VL, specific for the human CD3, with a 6× His tag as an optional Tag, with the fibritin domain as the oligomerizing domain, and containing a hydrophilic spacer section between the oligomerizing domain and HIV-Tat,
and
MALLLCFVLLCGVVDFARSLSASDIKLQQSGAELARPGASVKMSCKTSGYTFTRYTM HWVKQRPGQGLEWIGYINPSRGYTNYNQKFKDKATLTTDKSSSTAYMQLSSLTSEDS AVYYCARYYDDHYCLDYWGQGTTLTVSSGGGGSGGGGSGGGGSDIQLTQSPAIMSA SPGEKVTMTCRASSSVSYMNWYQQKSGTSPKRWIYDTSKVASGVPYRFSGSGSGTS YSLTISSMEAEDAATYYCQQWSSNPLTFGAGTKLELKEQKLISEEDLNMHTGHHHHH HPRGPTIKPCPPCKECHKCPAPNLEGGPSVFIFPPNIKDVLMISLTPKVTCVVVDVSED DPDVRISWFVNNVEVHTAQTQTHREDYNSTIRVVSALPIQHQDWMSGKEFKCKVNN KDLPSPIERTISKIKGLVRAPQVYILPPPAEQLSRKDVSLTCLVVGFNPGDISVEWTSNG HTEENYKDTAPVLDSDGSYFIYSKLDIKTSKWEKTDSFSCNVRHEGLKNYYLKKTISR TPGKGYIPEAPRDGQAYVRKDGEWVLLSTFLSPAGGGGSGGRKKRRQRRR
(SEQ ID NO: 4), containing the anti-CD3 scFV as VH-hinge-VL, specific for the human CD3, with a 6× His tag as an optional Tag, and with the fibritin domain as the oligomerizing domain, and containing a hydrophilic spacer section between the oligomerizing domain and HIV-Tat. Generally, arrangement of a hydrophilic spacer between the oligomerizing domain and HIV-Tat is optional, and preferred. The fibritin domain results in trimerization of the secreted fusion protein. It was found that the oligomerization of the fusion protein results in a higher efficacy of activation of T-cells compared to a fusion protein without oligomerization domain. As an alternative to the fibritin domain, GCN4, especially its human homolog, the activating transcription factor 4 (ATF4), can be contained in the fusion protein.

Generally, the fusion protein can be devoid of the Tag, as the Tag only serves as an epitope for an analytical antibody or, in the case of the 6× His tag, the Tag serves for enabling Ni-affinity purification of the fusion protein.

The hinge generally preferably has an amino acid sequence having sequence identity or homology of at least 80%, of at least 85%, of at least 90%, of at least 95% to at least one of amino acids 285 . . . 520 of SEQ ID NO: 1, amino acids 285 . . . 520 of SEQ ID NO: 2, amino acids 285 . . . 520 of SEQ ID NO: 3, and amino acids 285 . . . 520 of SEQ ID NO: 4.

The fibritin domain generally preferably has an amino acid sequence having sequence identity or homology of at least 80%, of at least 85%, of at least 90%, of at least 95% to at least one of amino acids 521 . . . 550 of SEQ ID NO: 2, and amino acids 521 . . . 550 of SEQ ID NO: 4.

The HIV-Tat domain, herein also termed protein transduction domain, generally preferably has an amino acid sequence having sequence identity or homology of at least 80%, of at least 85%, of at least 90%, of at least 95% to at least one of amino acids 521 . . . 530 of SEQ ID NO: 1, amino acids 557 . . . 566 of SEQ ID NO: 2, amino acids 521 . . . 530 of SEQ ID NO: 3, and amino acids 557 . . . 566 of SEQ ID NO: 4. For the purpose of this invention, the HIV-Tat domain also includes peptide sections of at least 5 amino acids or at least 6 amino acids, at least 7 amino acids, at least 8 amino acids, at least 9 amino acids, e.g. in each case up to 20 amino acids, e.g. up to 19 amino acids, up to 18 amino acids, up to 17 amino acids, up to 16 amino acids, up to 15 amino acids, up to 14 amino acids, up to 13 amino acids, up to 12 amino acids, up to 11 amino acids, up to 10 amino acids, preferably 5 to 20 amino acids or 9 to 20 amino acids.

The HIV-Tat domain for the purpose of the invention has at least 40%, preferably at least 50%, more preferably at least 60%, at least 70% or at least 80% amino acids, and/or at least 4 amino acids, at least 5, or preferably at least 6 amino acids, which in each case are independently selected from the group of Arg, Lys and His, e.g. consisting of Arginine, Lysine or both Arginine and Lysine, optionally including Histidine, e.g. the HIV-Tat domain for the purpose of the invention comprising or consisting of 6 to 9 Arginines, Lysines, or a combination of Arginines and Lysines, in each case optionally further containing Histidines, e.g. 1 to 5 Histidines. Further, the HIV-Tat domain for the purpose of the invention includes the herpes simplex virus protein VP22 (SEQ ID NO: 81) and the protein transduction domain of antennapedia homeodomain protein (AntP) (SEQ ID NO: 82). The HIV-Tat domain preferably has SEQ ID NO: 20.

These peptide sections for the purpose of the invention are included in the term HIV-Tat domain, because these peptide sections in the fusion protein after its secretion have the function to bind the fusion protein to cells which are in the vicinity of the secreting cells. Accordingly, the HIV-Tat domain as defined herein for the purpose of the invention can also be referred to as transduction domain.

The anti-CD3 scFv generally preferably has an amino acid sequence having a sequence identity or homology of its variable light chain of at least 80%, of at least 85%, of at least 90%, of at least 95% to one of the variable light chain sections of one of SEQ ID NO: 1, SEQ ID NO: 2, SEQ ID NO: 3 and SEQ ID NO: 4, and a sequence identity or homology of its variable heavy chain of at least 80%, of at least 85%, of at least 90%, of at least 95% to one of the variable heavy chain sections of one of SEQ ID NO: 1, SEQ ID NO: 2, SEQ ID NO: 3 and SEQ ID NO: 4.

Optionally, the fusion protein is encoded on a nucleic acid construct under the translational control of an IRES (internal ribosome entry site), which is a combination of an IRES and the fusion protein encoding nucleic acid construct is arranged in 3′ of a viral protein, e.g. E1B, such that transcription of the mRNA encoding the fusion protein is under control of a viral promoter, e.g. the E1B promoter, and translation of the fusion protein is dependent on the IRES.

Preferably, the nucleic acid construct encoding the fusion protein of the invention is arranged in expression units that avoid strong expression to overexpression of the fusion protein. A preferable weak expression of the fusion protein can be achieved by an expression unit that combines a viral (e.g. E1B) or a physiological promoter with an IRES motif for expression control of the fusion protein of the invention which leads to reduced expression levels of the gene located 3′ of the IRES motif. In the combination of a promoter with an IRES that controls the translation of the fusion protein encoding nucleic acid, the promoter can be any promoter, e.g. a strong, a medium, or a weak promoter, e.g. a physiological or viral promoter. An exemplary strong promoter is the EF1 alpha promoter. Alternatively, the fusion protein of the invention can preferably be expressed under direct control of a weak promoter, which in a human cell, preferably in a human tumour cell, has a lower activity than the strong physiological phosphoglycerate kinase (PGK) promotor, such as a promoter having the strength of a glycerol aldehyde-3-phosphate dehydrogenase promoter, or a tumor-specific promoter. Accordingly, the nucleic acid section encoding the fusion protein preferably is under direct control of a weak promoter, such as the phosphoenol pyruvate carboxykinase promoter, a glycerol aldehyde-3-phosphate dehydrogenase promoter, or a tumour-specific promoter, each preferably of human origin.

The sequence listing represents the amino acid sequences from N-terminus to C-terminus and encoding nucleic acid sequences from 5′ to 3′ of fusion proteins of the invention, in detail in SEQ ID NO: 1 a fusion protein (anti-CD3 h-TAT) having a secretory leader, an anti-CD3 scFv directed against human CD3 (anti-human CD3 scFv) in the arrangement of variable light chain-linker-variable heavy chain, a Myc-tag, a His-tag, a hinge domain, and HIV-Tat, SEQ ID NO: 2 a fusion protein (anti-CD3 h-TAT-Trimer) having a secretory leader, an anti-CD3 scFv directed against human CD3 (anti-human CD3 scFv) in the arrangement of variable light chain-linker-variable heavy chain, a Myc-tag, a His-tag, a hinge domain, a fibritin domain, a hydrophilic spacer, and HIV-Tat,
SEQ ID NO: 3 a fusion protein (anti-CD3 h-TAT) having a secretory leader, an anti-CD3 scFv directed against human CD3 (anti-human CD3 scFv) in the arrangement of variable heavy chain-linker-variable light chain, a Myc-tag, a His-tag, a hinge domain, and HIV-Tat, SEQ ID NO: 4 a fusion protein (anti-CD3 h-TAT-Trimer) having a secretory leader, an anti-CD3 scFv directed against human CD3 (anti-human CD3 scFv) in the arrangement of variable heavy chain-linker-variable light chain, a Myc-tag, a His-tag, a hinge domain, a fibritin domain, a hydrophilic spacer, and HIV-Tat,
SEQ ID NO: 5 the coding DNA for a fusion protein of SEQ ID NO: 1 (anti-CD3 h-TAT),
SEQ ID NO: 6 the coding DNA for a fusion protein of SEQ ID NO: 2 (anti-CD3 h-TAT-Trimer),
SEQ ID NO: 7 the coding DNA for a fusion protein of SEQ ID NO: 3 (anti-CD3 h-TAT), SEQ ID NO: 8 the coding DNA for a fusion protein of SEQ ID NO: 4 (anti-CD3 h-TAT-Trimer),
SEQ ID NO: 9 a fusion protein (anti-CD3m-TAT) having a secretory leader, an anti-CD3 scFv directed against murine CD3 (anti-murine CD3 scFv) in the arrangement of variable light chain-linker-variable heavy chain, a Myc-tag, a His-tag, a hinge domain, and HIV-Tat, SEQ ID NO: 10 a fusion protein (anti-CD3m-TAT-Trimer) having a secretory leader, an anti-CD3 scFv directed against murine CD3 (anti-murine CD3 scFv) in the arrangement of variable light chain-linker-variable heavy chain, a Myc-tag, a His-tag, a hinge domain, a fibritin domain, a hydrophilic spacer, and HIV-Tat,
SEQ ID NO: 11 the coding DNA for a fusion protein of SEQ ID NO: 9 (anti-CD3m-TAT),
SEQ ID NO: 12 the coding DNA for a fusion protein of SEQ ID NO: 10 (anti-CD3m-TAT),
SEQ ID NO: 13 the coding DNA for a preferred viral vector which encodes a fusion protein of the invention at nucleotides No. 4339 . . . 5919, which coding sequence for the fusion protein can be replaced by a coding sequence for any fusion protein of the invention,
SEQ ID NO: 71 the coding DNA for a preferred viral vector which encodes a fusion protein of the invention at nucleotides No. 4339 . . . 6027 in the embodiment containing a fibritin domain as a trimerization domain, which coding sequence for the fusion protein can be replaced by a coding sequence for any fusion protein of the invention.

An oncolytic viral particle is e.g. a herpes simplex virus, a reovirus, Newcastle disease virus, vesicular stomatitis virus, parvovirus H1, measles virus, vaccinia virus, maraba virus, polio virus, coxsackievirus, in each case attenuated and/or inactivated, preferably an adenovirus, especially an adenovirus serotype 5, with a deleted or inactivated gene E1B55k and/or deleted or inactivated gene E1B19k, the inactivation being introduced e.g. by mutation, an adenovirus with a deletion in the Rb binding domain of E1A, also referred to as delta24 adenovirus or delta 922-947 adenovirus, adenovirus containing a tumour specific promoter controlling expression of E1A, which is e.g. the telomerase promoter, the AFP promoter, PSA promoter, DF1/Muc promoter, tyrosinase promoter, a HIF1alpha-dependent promoter, or a E2F/C-myc-dependent promoter.

A preferred oncolytic virus is a viral particle according to WO2021/239586 A1, the contents of which are incorporated herein by reference. A preferred embodiment of an oncolytic viral particle encoding a fusion protein of the invention is an adenovirus, which preferably contains the nucleic acid sequence of SEQ ID NO: 13, wherein the sequence encoding the fusion protein of nucleotides No. 4405-5985, which contains an anti-murine CD3 scFv, can optionally be replaced by a sequence encoding the fusion protein having an anti-human CD3 scFv, e.g. one of the embodiments of the fusion protein containing the anti-human CD3-scFv-linker-HIV-Tat, optionally with an oligomerization domain and/or with a hydrophilic spacer between the linker and the HIV-Tat domain. Preferably, the adenovirus encoding DNA from 5′ to 3′ comprises or consists of a left-end inverted terminal repeat (L-ITR), a first promoter, a coding sequence for E1A, an E1B promoter controlling a coding sequence for E1B, an expression cassette for at least one effector molecule, preferably an expression cassette encoding inhibitory RNA (RNAi) that are specific for RNAi target sites within the E1A encoding sequence and/or within the E1B promoter and/or within the E1B encoding sequence, vector backbone sequence, and on the opposite DNA strand an expression cassette for E4 comprising the orf4, the expression cassette for E4 containing in functional relationship a promoter driving expression of E4, and a right-end inverted terminal repeat (R-ITR). The arrangement of the L-ITR, E1A, a promoter controlling expression of E1B, preferably of an expression cassette for at least one effector molecule on one DNA strand, and preferably of coding sequences for inhibitory RNA under the control of a promoter which is activated by the presence of p53, e.g. the prMinRGC promoter, on one DNA strand, and arrangement of the expression cassette for E4 on the opposite DNA strand corresponds to the arrangement of the R-ITR, E4-promoter, and of the orfs constituting the coding sequence for E4 from 5′ to 3′ on the opposite strand. The expression cassette for an E4-orf4 encoded protein is arranged on the DNA strand opposite to the strand encoding the L-ITR, the first promoter, the E1A encoding sequence and the E1B promoter and the E1B encoding sequence. The L-ITR is arranged at one terminus of the DNA and the R-ITR is arranged at the terminus of the DNA opposite of the L-ITR.

The first promoter preferably is CMVs (nucleotides No. 380-625 of SEQ ID NO: 13), which has the advantage of becoming activated following infection of a cell with a time delay that allows for the effective inhibition of the E1A encoding sequence, the E1B promoter and of the E1B encoding sequence by inhibitory RNA molecules that are expressed under the control of prMinRGC.

Preferably, the coding sequence for E1A, the E1B promoter, and the coding sequence for E1B contain RNAi target sites for binding inhibitory RNA, e.g. shRNA, and the adenovirus encoding DNA contains an expression cassette for inhibitory RNA, e.g. for shRNA, which bind to the RNAi target sites, which expression cassette preferably is under the control of a promoter which is activated by presence of p53, e.g. the promoter prMinRGC. The coding sequences for inhibitory RNA, which inhibit expression of the adenoviral genes, e.g. of E1A, E1B, E4, pTP, Pol, are preferably expressed under the control of the prMinRGC promoter. The prMinRGC promoter is preferred as the promoter which is activated by the presence of p53.

Preferably, the viral particle is encoded by and contains a nucleic acid sequence of SEQ ID NO: 13 or of SEQ ID NO: 71.

As an alternative to a viral particle, a nucleic acid construct encoding the fusion protein can be contained in a liposome used as a carrier or the nucleic acid construct can be associated with a synthetic carrier particle. Generally preferred, a carrier containing a nucleic acid construct encoding the fusion protein is provided in a formulation suitable for direct injection into tumour tissue, and such a carrier is preferably used for direct injection into tumour tissue.

Generally, the fusion protein is a soluble protein, i.e. it is devoid of a transmembrane domain, and the fusion protein is secreted from the cell that contains a nucleic acid construct encoding the fusion protein.

The invention is now described by examples with reference to the figures, which show in

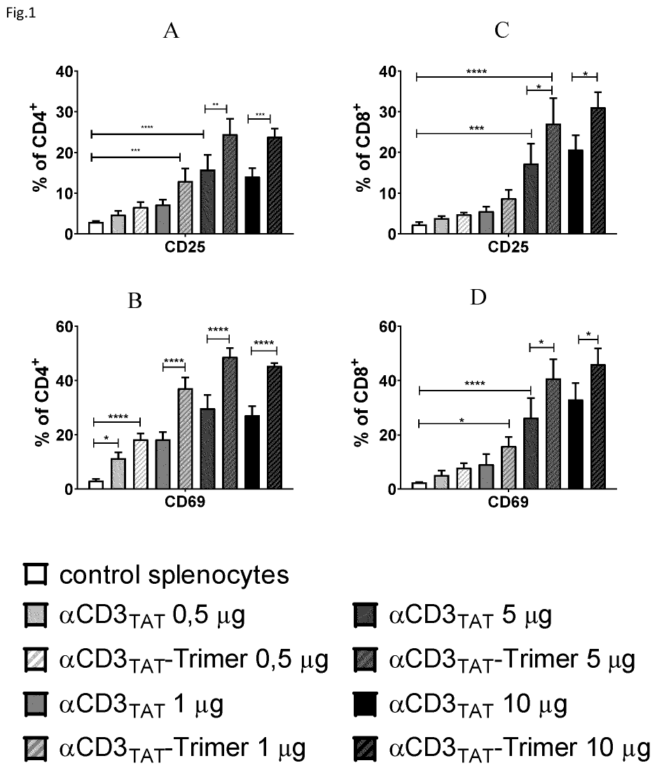

FIG. 1A-1D flow cytometry (FACS) results for the activation markers CD25 and CD69 on murine CD4+ T-cells and on CD8+ T-cells for CD90.2+ leukocytes in presence of fusion protein of the invention,

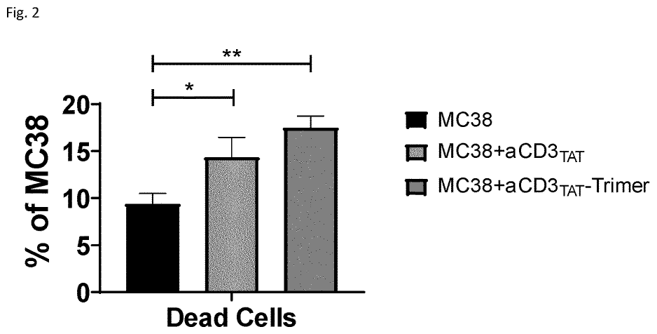

FIG. 2 killing rates of murine cancer cells by T-cells in presence of fusion protein of the invention,

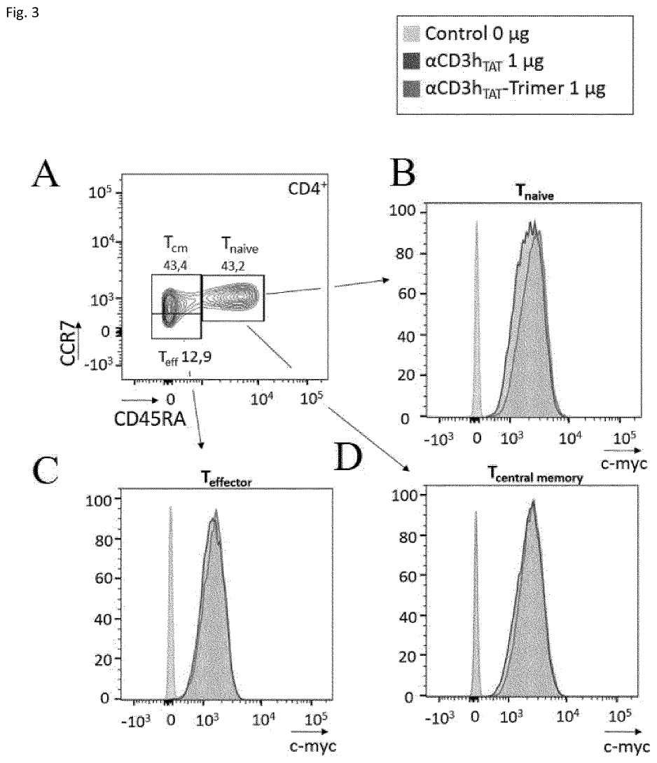

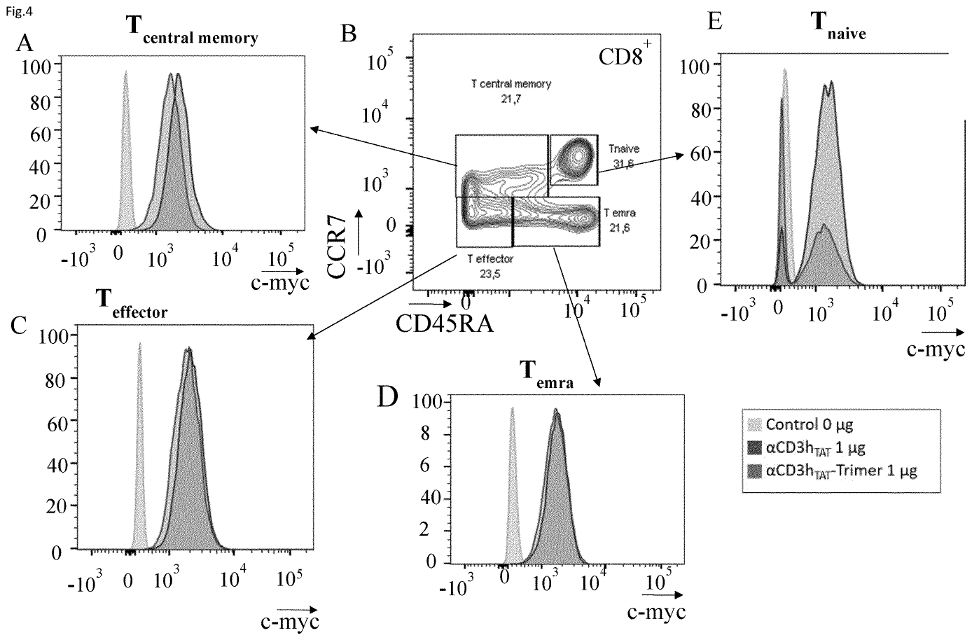

FIG. 3A-3D data for binding of human CD4+ T-cells by the fusion protein of the invention,

FIG. 4A-4E data for binding of human CD8+ T-cells by the fusion protein of the invention,

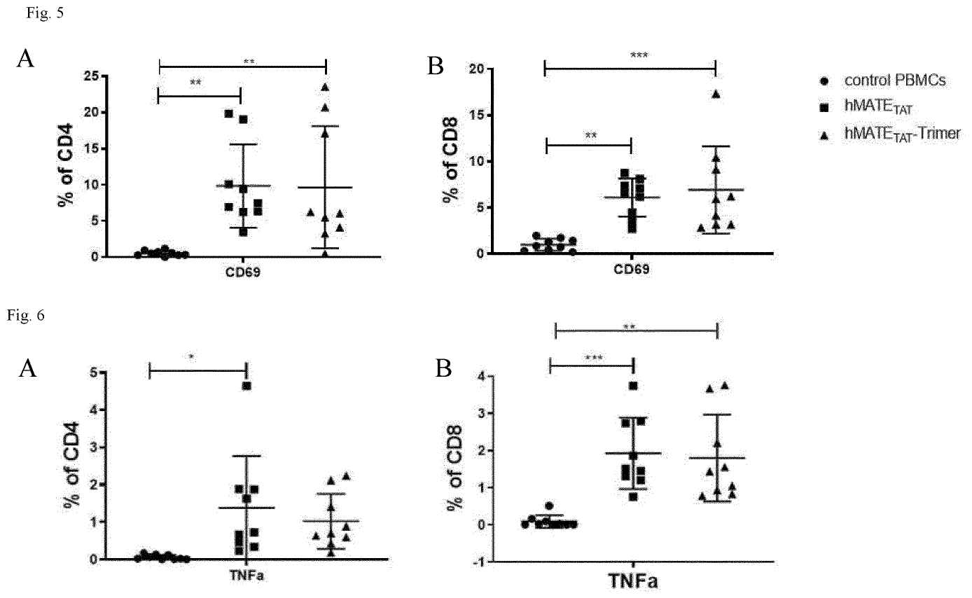

FIG. 5A-5B data for activation of both human CD4+ and CD8+ T-cells for anti-CD69 antibody staining by embodiments of the fusion protein,

FIG. 6A-6B by intracellular cytokine staining for TNFα cytokine production by human CD4+ and CD8+ T-cells incited by the fusion protein,

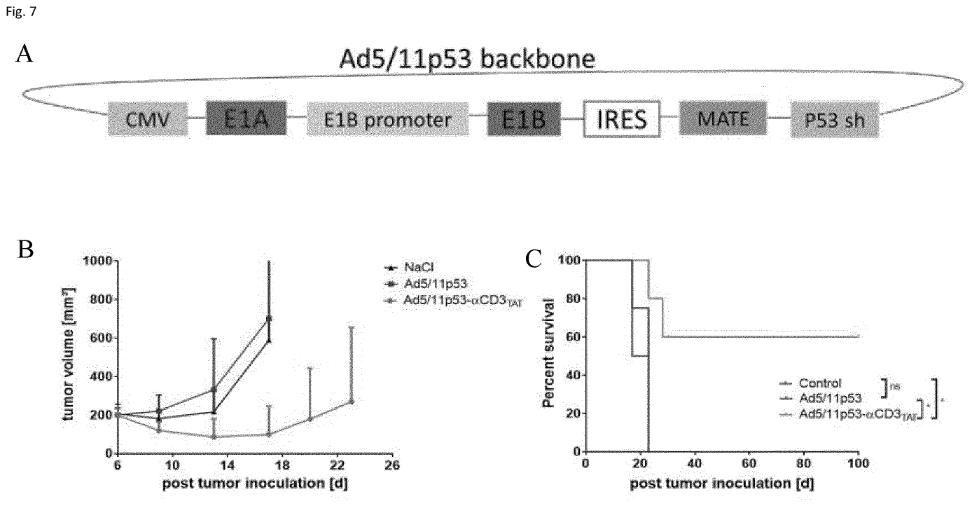

FIG. 7A a schematic of a nucleic acid sequence encoding preferred a viral particle with a fusion protein of the invention,

FIG. 7B data for increased tumour regression in mice caused by expression of the fusion protein of the invention,

FIG. 7C data for improved survival of tumour bearing mice caused by expression of the fusion protein of the invention,

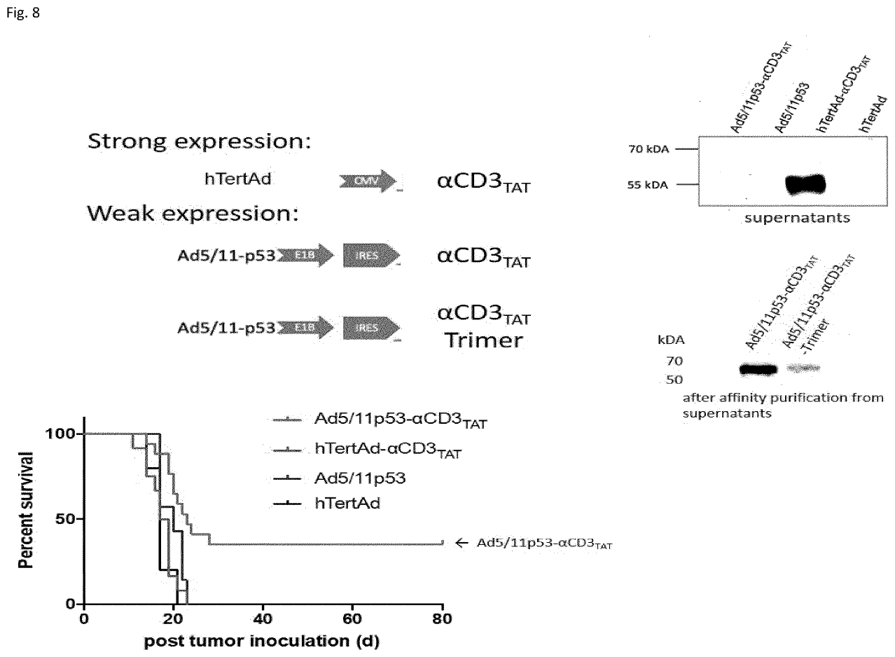

FIG. 8A a schematic of nucleic acid constructs for expression of the fusion protein of the invention,

FIG. 8B Western blots of culture supernatants of 293 T-cells expressing fusion proteins of the invention under the control of a weak or of a strong promoter,

FIG. 8C data for improved survival of tumour bearing mice caused by expression of the fusion protein of the invention,

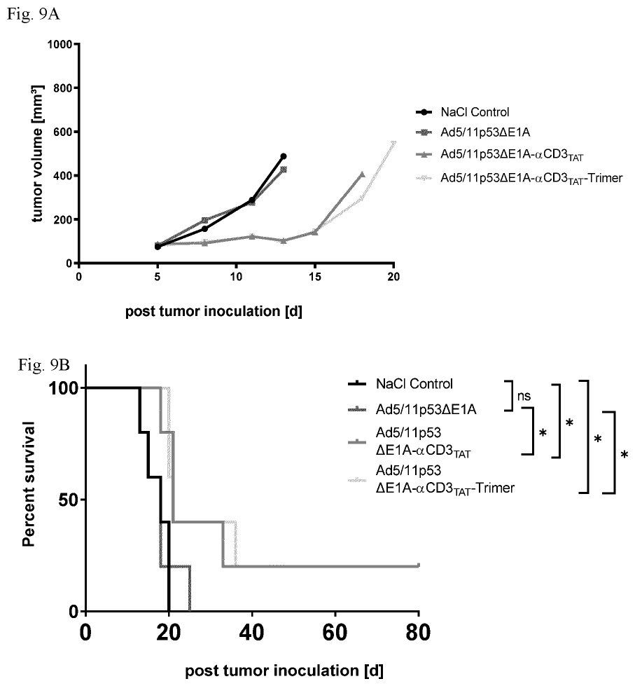

FIG. 9A data for increased tumour regression in mice caused by expression of the fusion protein of the invention,

FIG. 9B data for improved survival of tumour bearing mice caused by expression of the fusion protein of the invention,

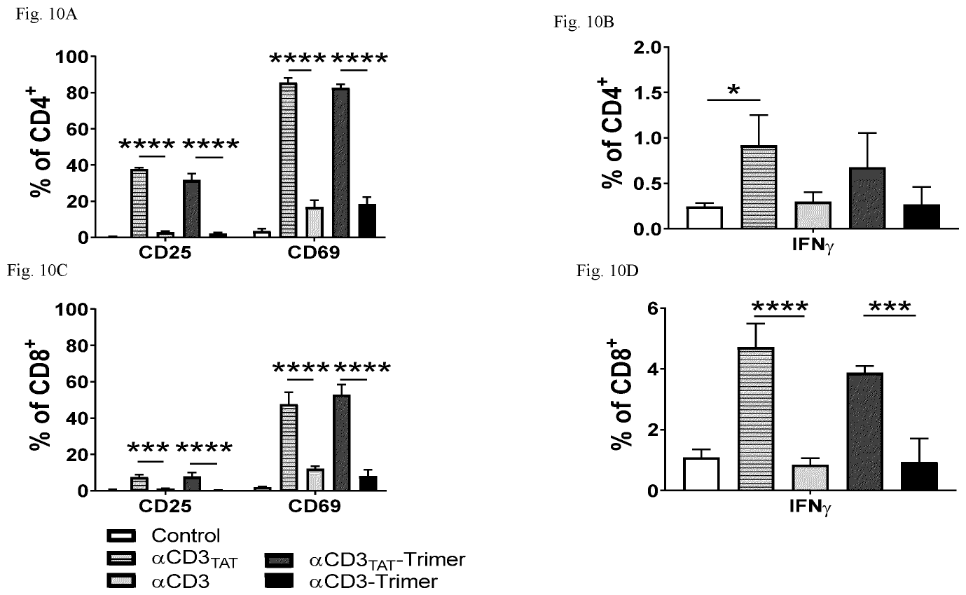

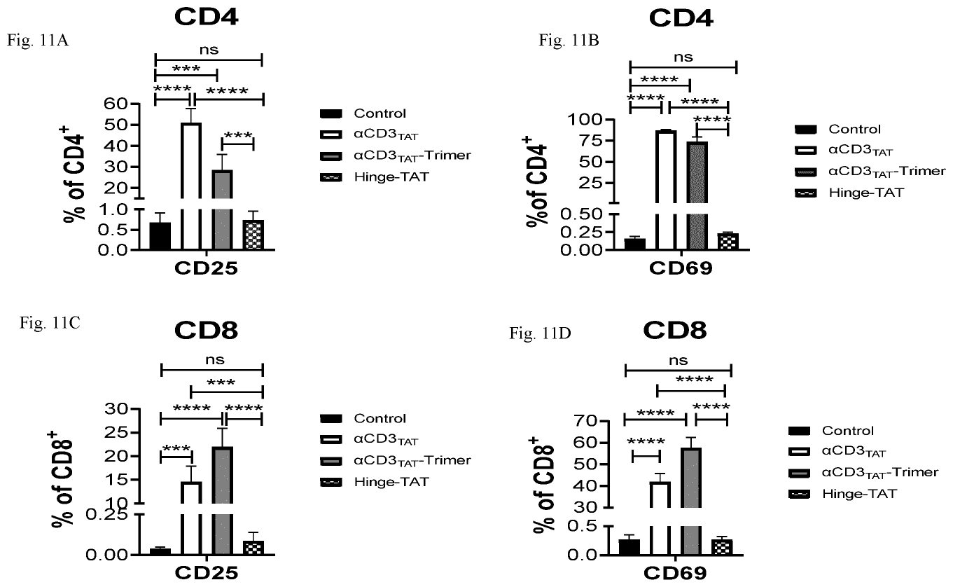

FIG. 10A data for activation markers of murine splenocytes after addition of fusion protein of the invention or with comparative protein lacking the TAT-protein transduction domain,

FIG. 10B data for activation markers of murine splenocytes after addition of fusion protein of the invention or with comparative protein lacking the TAT-protein transduction domain,

FIG. 10C data for activation markers of murine splenocytes after addition of fusion protein of the invention or with comparative protein lacking the TAT-protein transduction domain,

FIG. 10D data for activation markers of murine splenocytes after addition of fusion protein of the invention or with comparative protein lacking the TAT-protein transduction domain,

FIG. 11A data for activation markers of splenocytes after addition of fusion protein of the invention or with comparative protein lacking the anti-CD3-domain,

FIG. 11B data for activation markers of splenocytes after addition of fusion protein of the invention or with comparative protein lacking the anti-CD3-domain,

FIG. 11C data for activation markers of splenocytes after addition of fusion protein of the invention or with comparative protein lacking the anti-CD3-domain,

FIG. 11D data for activation markers of splenocytes after addition of fusion protein of the invention or with comparative protein lacking the anti-CD3-domain,

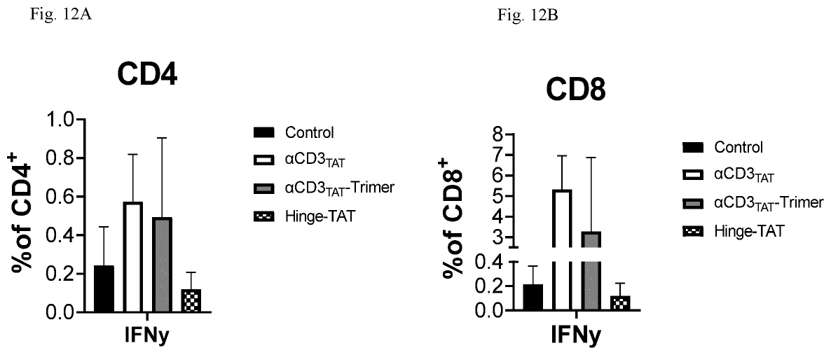

FIG. 12A data for functional activation markers of splenocytes after addition of fusion protein of the invention or with comparative protein lacking the anti-CD3-domain,

FIG. 12B data for activation markers of splenocytes after addition of fusion protein of the invention or with comparative protein lacking the anti-CD3-domain,

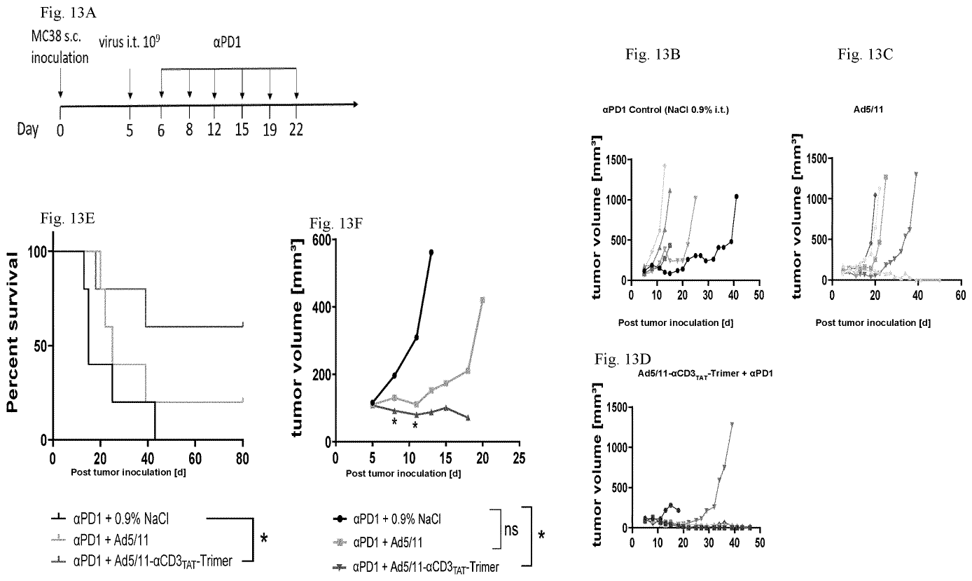

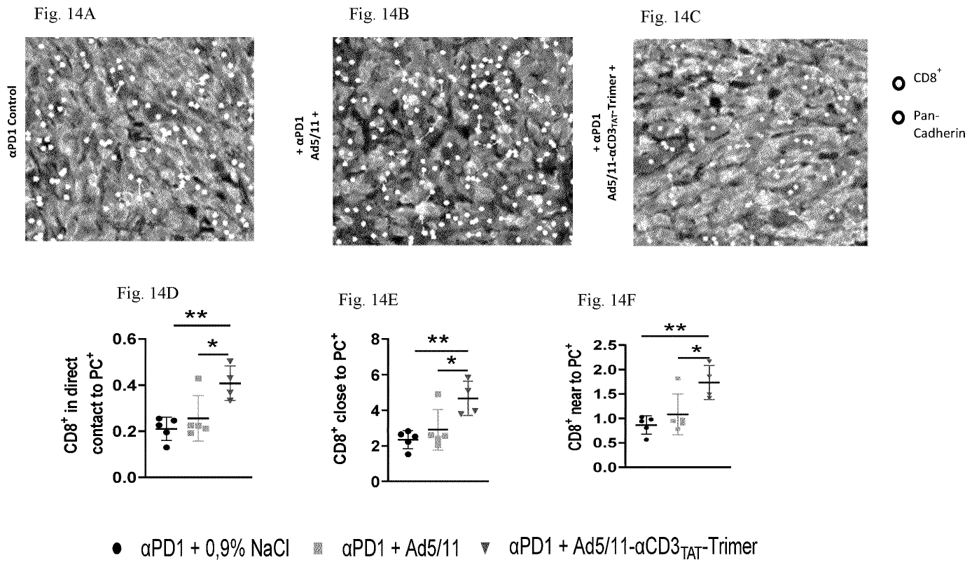

FIG. 13A a schematic of in vivo tumour treatment by viral particles expressing a fusion protein of the invention and subsequent applications of an anti-PD1 antibody,

FIGS. 13B, 13C, and 13D individual tumour sizes for comparative treatments, including a treatment using a virus expressing a fusion protein of the invention

FIG. 13E survival rates and FIG. 13F shows mean tumour sizes of these animals,

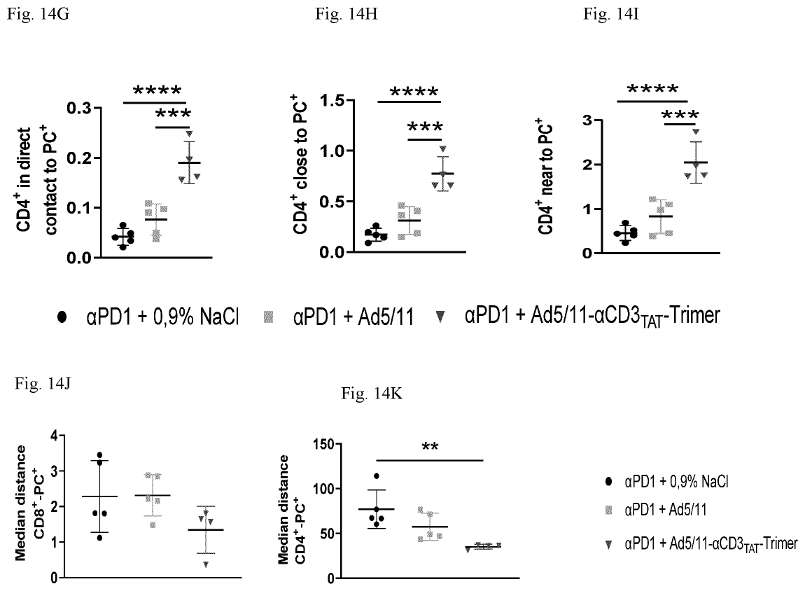

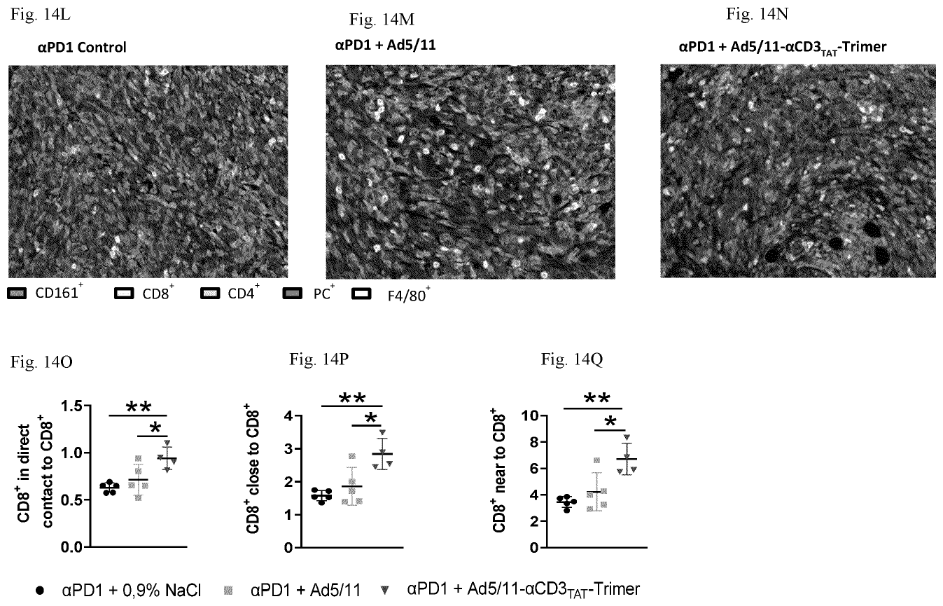

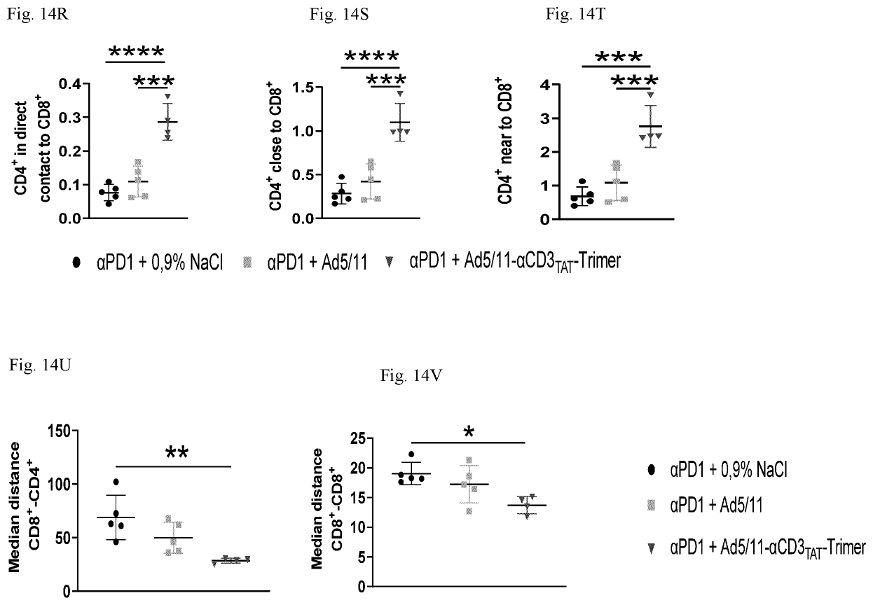

FIG. 14A a micrograph of tumour tissue after treatment with αPD1 only,

FIG. 14B a micrograph of tumour tissue after treatment with αPD1 and empty oncolytic virus,

FIG. 14C a micrograph of tumour tissue after treatment with αPD1 and oncolytic virus expressing a fusion protein of the invention,

FIG. 14D, 14E, 14F results of spatial analyses of CD8+ T-cells with respect to tumour cells (PC+),

FIG. 14G, FIG. 14H and FIG. 14I results of spatial analyses of CD4+ cells with respect to tumour cells (PC+),

FIG. 14J median distances of CD8+ T-cells from tumour cells,

FIG. 14K median distances of CD4+ T-cells from tumour cells,

FIG. 14L a micrograph of tumour tissue after treatment with αPD1-antibody only,

FIG. 14M a micrograph of tumour tissue after treatment with PD1-antibody and empty oncolytic virus,

FIG. 14N a micrograph of tumour tissue after treatment with αPD1-antibody in combination with oncolytic virus encoding a fusion protein of the invention,

FIG. 14O percentages of CD8+ T-cells in direct contact with other CD8+ T-cells,

FIG. 14P percentages of CD8+ T-cells in close distance to other CD8+ T-cells,

FIG. 14Q percentages of CD8+ T-cells which are near to other CD8+ T-cells,

FIG. 14R percentages of CD4+ T-cells in direct contact with CD8+ T-cells,

FIG. 14S percentages of CD4+ T-cells in close distance to CD8+ T-cells,

FIG. 14T percentages of CD4+ T-cells which are near to CD8+ T-cells,

FIG. 14U median distances of CD8+ T-cells to CD4+ T-cells, and

FIG. 14V median distances between CD8+ T-cells

In the figures, * denotes significance, ** higher significance, *** even more pronounced significance, n.s. denotes no significance. In the examples, the viral particles were based on the oncolytic adenovirus Ad5/11 carrying the deletion of AELA and the p53-dependent expression of RNAi, essentially leading to expression of viral protein in p53-deficient tumour cells and limited levels of viral protein in p53-normal cells. This preferred viral particle is encoded by SEQ ID NO: 13 or consists thereof, wherein the coding sequence for the fusion protein of nucleotides 4405-5985 can be replaced by a nucleotide sequence encoding an embodiment of the fusion protein of the invention, e.g. encoding one of SEQ ID NO: 1, SEQ ID NO: 2, SEQ ID NO: 3 and SEQ ID NO: 4.

### EXAMPLE 1: ACTIVATING T-CELLS BY PRESENCE OF THE FUSION PROTEIN IN VITRO

As an example for a fusion protein without oligomerizing domain, a fusion protein consisting of secretory leader peptide-murine anti-CD3-scFv-hinge-HIV-Tat and, separately as an example for a fusion protein containing an oligomerizing domain a fusion protein consisting of secretory leader peptide-murine anti-CD3-scFv-Tag-hinge-oligomerizing domain-HIV-Tat were produced in HEK-293 cells. These fusion proteins had an amino acid sequence corresponding to SEQ ID NO: 9 as e.g. encoded by SEQ ID NO: 11, or SEQ ID NO: 10 as e.g. encoded by SEQ ID NO: 12, respectively, containing an anti-murine CD3 scFv instead of the anti-human CD3 scFv of SEQ ID NO: 1 and 3.

In short, HEK-293 cells were cultivated and transfected by using polyethyleneimine (PEI) with a nucleic acid construct comprising SEQ ID NO: 11 or SEQ ID NO: 12, encoding one of the fusion proteins and then cultivated for 72 h. The fusion proteins were isolated from the cell culture supernatant by Ni-affinity chromatography using a Ni-NTA column. The purified fusion proteins were added to separate aliquots of freshly isolated murine splenocytes and incubated under cell culture conditions for 24 h. In short, splenocytes were isolated from spleens of mice and one of the fusion proteins was added at amounts of 0.5 μg, 1 μg, 5 μg or 10 μg, respectively for 1 mL splenocyte culture medium.

Subsequent to the incubation of splenocytes with the fusion protein, the splenocytes were analyzed by flow cytometry (FACS) for the activation markers CD25 and CD69 on CD4+ T-cells and on CD8+ T-cells for CD90.2+ leukocytes. The results are depicted in FIGS. 1A and 1B for CD4+ T-cells and in FIGS. 1C and 1D for CD8+ T-cells, for the fusion protein without oligomerizing domain (αCD3_{TAT}), and for the fusion protein with the fibritin oligomerizing domain (αCD3_{TAT}-Trimer). The results show that the percentage of both CD4+ and CD8+ T-cells having the activated CD25+ and CD69+ increased with increasing concentrations of added fusion protein. Further, it is shown that the trimerizing embodiment of the fusion protein, which contains the fibritin oligomerization domain, at the same protein concentration induced an increased activation of both CD4+ and CD8+ T-cells.

Further, 1×10⁶ freshly isolated murine splenocytes supplemented with one of the fusion proteins consisting of secretory leader peptide-anti-murine CD3-scFv-hinge-HIV-Tat or secretory leader peptide-anti-murine CD3-scFv-Tag-hinge-oligomerizing domain-HIV-Tat at a total amount of 1 μg in a total volume of 1 mL culture medium were added to 1×10⁵ murine colon cancer cells (MC38) resulting in an effector: target cell ratio of 10:1. After 24 h co-incubation under cell culture conditions, the number of surviving cancer cells was determined by flow cytometry. FIG. 2 shows that both fusion proteins resulted in an increased killing of cancer cells, and that the fusion protein containing the oligomerization domain resulted in increased killing of tumour cells. For the MC38 tumour cells, the fusion protein containing the fibritin oligomerization domain (FIG. 2: MC38+αCD3_{TAT}-Trimer) was used at the same protein concentration as used for the embodiment without the oligomerizing domain (secretory leader peptide-anti-murine CD3-scFv-hinge-HIV-Tat, FIG. 2: MC38+αCD3_{TAT}). Accordingly, this effective activation of T-cells showed a significantly higher molar activating effect for the embodiment containing an oligomerizing domain.

### EXAMPLE 2: ACTIVATION OF HUMAN T-CELLS FROM PBMC BY A FUSION PROTEIN

Human T-cells were isolated from human whole blood as PBMC by pancoll gradient centrifugation as a buffy coat layer. The activation of the human T-cells was analysed by determining the CD69 as an activation marker by FACS on CD4+ and CD8+ T-cells after incubation for 24 h of PBMC in 1 mL cell culture medium with 1 μg fusion protein consisting of secretory leader peptide-anti-human CD3-scFv-hinge-HIV-Tat (SEQ ID NO: 1, αCD3h_{TAT}, or alternatively SEQ ID NO: 3) or secretory leader peptide-anti-human CD3-scFv-Tag-hinge-oligomerizing domain-HIV-Tat (SEQ ID NO: 2, αCD3h_{TAT}-Trimer, or alternatively SEQ ID NO: 4). During FACS, in addition to a labelled anti-CD69 antibody, labelled anti-CD4 antibody, anti-CD8 antibody, anti-CCR7 antibody and anti-CD45RA antibody were used, TNFα was determined by intracellular immunologic cytokine staining (ICS), and anti-c-myc antibody was used for specific staining of the fusion protein.

The FACS results depicted in FIG. 3A-D show that subtypes of human CD4+ T-cells, specifically naïve T-cells, T effector, and central memory T-cells were associated with the fusion protein as shown by binding of anti-c-myc antibody.

The FACS results depicted in FIG. 4A-E show that also subtypes of human CD8+ T-cells, specifically naïve T-cells, T effector, TEMRA, and central memory T-cells were associated with the fusion protein as shown by binding of anti-c-myc antibody.

The FACS results depicted in FIG. 5A-B for anti-CD69 antibody staining show that both CD4+ and CD8+ T-cells were activated by each embodiment of the fusion proteins, consisting of secretory leader peptide-anti-human-CD3-scFv-hinge-HIV-Tat (SEQ ID NO: 1, hMATE_{TAT}) or secretory leader peptide-murine anti-CD3-scFv-hinge-HIV-Tat or secretory leader peptide-anti-human-CD3-scFv-Tag-hinge-oligomerizing domain-HIV-Tat (SEQ ID NO: 2, hMATE_{TAT}-Trimer).

The FACS results depicted in FIG. 6A-B for intracellular cytokine staining for TNFα (TNFa) shows cytokine production by CD4+ and CD8+ T-cells activated by one of the fusion proteins.

In summary, these results show that the fusion protein of the invention effectively binds to CD4+ and to CD8+ T-cells, including several subtypes of the T-cells, and that binding of the fusion protein strongly activates both CD4+ and CD8+ T-cells, including activating production of cytotoxic cytokines.

### EXAMPLE 3: TREATMENT OF A SOLID TUMOUR BY ONCOLYTIC ADENOVIRUS EXPRESSING THE FUSION PROTEIN

As a generally preferred example for an oncolytic virus, also referred to as oncolytic viral particle, an adenovirus serotype 5 (Ad5/11-p53) was used, which in dependence on presence of p53 in normal, non-cancer cells expresses shRNA directed against binding sites in its nucleic acid construct that are arranged to avoid expression of viral genes in normal cells, while allowing expression of encoded genes and replication in tumour cells that have lower expression of p53. Further, this oncolytic virus carried an inactivated E4 orf3 locus and a mutated E4 orf4. A preferred embodiment of this oncolytic virus is encoded by SEQ ID NO: 13, which encodes a fusion protein of the invention in the embodiment without oligomerization domain. This oncolytic virus was found to be specific for tumour cells, especially for use in the treatment of a tumour. A preferred oncolytic viral particle is adenovirus Ad5/11, which contains a nucleic acid sequence encoding the fusion protein, in this case encoding the fusion protein containing an anti-murine CD3 scFv in order to bind to the T cells of the experimental mouse. In this example, the fusion protein consisted of secretory leader peptide-anti-murine-CD3-scFv-hinge-HIV-Tat as an exemplary embodiment. A schematic of the viral particle encoding nucleic acid sequence is depicted in FIG. 7A, showing that the fusion protein encoding sequence is arranged under the translational control of an IRES and transcriptionally controlled by the E1B promotor. As generally preferred, this viral particle in dependence on presence of p53 generates shRNA that inhibit replication of the viral particle itself, e.g. in healthy tissue that expresses p53.

As an example for treatment of a solid tumour, immune-competent C57BL/6 mice were subcutaneously injected into the right flank with 1×10⁷ syngeneic colon adenocarcinoma cells (MC38), and mice were kept for 6 d, e.g. until tumour growth of 200 mm³ was detected.

For comparison to viral particles (Ad5/11p53-αCD3_{TAT}) according to SEQ ID NO: 13, saline (NaCl) as a negative control, or viral particles without the fusion protein (Ad5/11p53) were used, corresponding to SEQ ID NO: 13 but without the coding sequence for the fusion protein (i.e. without nucleotides No. 4405-5985 of SEQ ID NO: 13). The adenovirus containing the DNA of SEQ ID NO: 13 and the comparative adenovirus were separately produced, e.g. in cultivated HEK293 cells. These viral particles were isolated from the culture supernatant and intratumorally injected at a single dose of 1×10⁹.

FIG. 7B shows the tumour volume in mice, demonstrating that the comparative adenovirus (Ad5/11p53) resulted in decreased tumour growth compared to saline (NaCl), and that the viral particle encoding a fusion protein of the invention (Ad5/11p53-αCD3_{TAT}) resulted in drastically lower tumour growth, also in comparison to the comparative adenovirus.

FIG. 7C shows the survival of tumour-bearing mice after treatment with saline (Control), with the comparative adenovirus (Ad5/11p53) that does not encode the fusion protein, and treatment with an otherwise identical viral particle (SEQ ID NO: 13, Ad5/11p53-αCD3_{TAT}) encoding the fusion protein.

The comparison of the anti-tumour effect of the viral particle encoding the fusion protein to the comparative adenovirus shows that the fusion protein significantly increases the anti-tumour activity of the viral particle.

### EXAMPLE 4: TREATMENT OF A SOLID TUMOUR BY ONCOLYTIC ADENOVIRUS EXPRESSING THE FUSION PROTEIN UNDER THE CONTROL OF A WEAK PROMOTER

As a generally preferred example for an oncolytic virus, also referred to as oncolytic viral particle, an adenovirus serotype 5, encoding a fusion protein of the invention, represented by SEQ ID NO: 13 Ad5/11p53-αCD3_{TAT} was used for testing the influence of the promoter strength driving transcription of the fusion protein encoding nucleic acid sequence. As a representative of a strong promoter, the CMV promoter was arranged to control transcription of the fusion protein encoding DNA section, replacing the IRES in SEQ ID NO: 13 arranged in 5′ to the fusion protein encoding DNA section. An expression unit comprising an IRES-motif arranged in 5′ of the fusion protein encoding nucleic acid section was used to achieve a weak expression similar to the use of a weak promoter controlling transcription of the fusion protein encoding DNA section as shown in SEQ ID NO: 13. As a further example, the fusion protein encoding DNA section in SEQ ID NO: 13 was replaced by a section encoding a fusion protein containing an oligomerization domain according to amino acid SEQ ID NO: 2 or SEQ ID NO: 4, in each embodiment under the control of the IRES.

FIG. 8A schematically depicts the nucleic acid constructs, for strong expression containing the CMV promoter (CMV, hTertAd) or for weak expression containing the IRES (Ad5/11-p53), controlling transcription of the fusion protein without oligomerization domain, the fusion protein consisting of secretory leader-anti-human CD3 scFv-MycTag-HisTag-hinge-HIV-Tat (αCD3_{TAT}, e.g. SEQ ID NO: 1 or SEQ ID NO: 3), or under the control of the weak promoter controlling transcription of the fusion protein containing an oligomerization domain, the fusion protein consisting of secretory leader-anti-human CD3 scFv-MycTag-HisTag-hinge-fibritin domain-hydrophilic spacer-HIV-Tat (αCD3_{TAT}Trimer, e.g. SEQ ID NO: 2 or SEQ ID NO: 4), wherein the fibritin domain represents an oligomerization domain and wherein the hydrophilic spacer is optional. A preferred embodiment of this oncolytic virus is encoded by SEQ ID NO: 71, which encodes a fusion protein of the invention in the embodiment containing a fibritin domain as an oligomerization domain.

FIG. 8B, upper picture, shows a Western blot of culture supernatants of HEK 293 cells at 48 h after infection by the viral particles encoding a fusion protein of the invention, with immune detection using a labelled anti-Myc antibody. For a sample of 50 μL protein of each supernatant, the Western blot shows in lane 1 the viral particle encoding the fusion protein under the control of the E1B promoter and an IRES-motif for expression of the fusion protein (Ad5/11p53-αCD3_{TAT}) of the invention, lane 2 as control a similar viral particle without encoding a fusion protein of the invention (Ad5/11p53), lane 3 the viral particle encoding the fusion protein under direct control of a strong promoter, represented by the CMV promoter (hTertAd-αCD3_{TAT}), lane 4 as a control the similar viral particle containing a strong promoter (CMV promoter) without encoding a fusion protein of the invention (hTertAd). This shows that the viral particle encoding the fusion protein under the control of a strong promoter yields high expression of the fusion protein, whereas in this experiment, expression of the fusion protein under the control of the E1B promoter and an IRES motiv was not sufficient for direct detection in the supernatant indicating a low expression level.

FIG. 8B, lower picture, shows a Western blot of protein samples obtained from cell culture supernatants after protein purification by metal ion affinity chromatography for targeted enrichment of fusion proteins. Supernatants were obtained from 293 T-cells at 48 h after infection by the viral particles encoding a fusion protein of the invention, and immune detection was performed using a labelled anti-Myc antibody. For a sample of 50 μL of the purified protein, lane 1 (unlabeled) shows the purified protein the of an empty virus (Ad5/11p53) lacking expression of a fusion protein, lane 2 shows protein purified after infection with the viral particle encoding the fusion protein under the control of the E1B promoter/IRES (Ad5/11p53-αCD3_{TAT}) of the invention, lane 3 for the viral particle encoding the fusion protein containing an oligomerization domain under the control of the E1B promoter/IRES (Ad5/11p53-αCD3_{TAT}-Trimer) of the invention.

This positive detection after concentrating the fusion protein of the invention proves that the fusion protein of the invention is present in soluble form in supernatants of infected cells when expressed under the control of a weak expression unit (E1B promoter linked with an IRES), both for the embodiment without an oligomerization domain, and for the embodiment containing an oligomerization domain.

FIG. 8C shows the survival rate of C57BL/6 mice bearing the MC38 tumour as described in Example 3, with intratumoral injection of these viral particles. The result shows that the expression of a fusion protein of the invention under the control of a weak promoter results in a drastically improved survival, i.e. tumour treatment, e.g. in comparison to treatment with an oncolytic viral particle encoding the same fusion protein under the control of a strong promoter and in comparison to the oncolytic viral particle without the fusion protein (SEQ ID NO: 13 lacking the nucleic acid sequence encoding the fusion protein).

### EXAMPLE 5: TREATMENT OF A SOLID TUMOUR BY ONCOLYTIC ADENOVIRUS EXPRESSING THE FUSION PROTEIN

As a generally preferred example for an oncolytic virus, also referred to as oncolytic viral particle, an adenovirus serotype 5, encoding a fusion protein of the invention, represented by SEQ ID NO: 13 Ad5/11p53 was used for testing, encoding a fusion protein of the invention without oligomerization domain (αCD3_{TAT}) and in an embodiment of the fusion protein containing an oligomerization domain (CD3_{TAT}-Trimer). C57BL/6 mice bearing the MC38 tumour were used as described in Example 3.

The viral particles were based on the oncolytic adenovirus Ad5/11 carrying the deletion of ΔE1A and the p53-dependent expression of RNAi according to SEQ ID NO: 13, encoding a fusion protein without oligomerization domain e.g. according to SEQ ID NO: 9 (Ad5/11p53ΔE1A-αCD3_{TAT}), or encoding a fusion protein that includes an oligomerization domain e.g. according to SEQ ID NO: 10 (Ad5/11p53ΔE1A-&CD3_{TAT}-Trimer). As a negative control, 0.9% saline (NaCl) and the viral particle without coding sequence for a fusion protein (Ad5/11p53ΔE1A) were used.

FIG. 9A shows that the tumour volume was significantly lower for viral particles expressing one of the fusion proteins of the invention, and FIG. 9B shows that survival, i.e. tumour treatment were significantly improved for viral particles expressing one of the fusion proteins of the invention, with a better effect for the fusion protein containing the oligomerization domain.

### EXAMPLE 6: ACTIVATION OF SPLENOCYTES IN VITRO

As a representative fusion protein consisting of secretory leader peptide-murine anti-CD3 scFv-hinge-HIV-Tat according to SEQ ID NO: 1, wherein the HIV-Tat domain was the protein transduction domain of HIV-Tat (Tat-PTD) according to SEQ ID NO: 20 was used, and in comparison a fusion protein consisting of secretory leader peptide-murine anti-CD3 scFv-hinge, corresponding to amino acids No. 1 . . . 520 of SEQ ID NO: 1, i.e. lacking the protein transduction domain. As a further comparison, a trimerized variant of the fusion protein of the invention was tested in comparison to the trimerized scFv.

In short, freshly isolated murine splenocytes were incubated with 1 μg of purified fusion protein consisting of secretory leader peptide-murine anti-CD3 scFv-hinge-HIV-Tat (MATE) or with the comparative fusion protein lacking the protein transduction domain (PTD). After 24 h of incubation, T-cells were immune-stained for the activation markers CD25 and CD69 and measured by flow cytometry, expression of IFNγ was assayed by ICS.

The results are depicted in FIG. 10, showing that splenocytes without added protein (control) were not activated, i.e. considered background only, whereas the fusion protein of the invention (αCD3_{TAT}) containing the PTD of HIV-Tat has very significantly higher activation activity, in comparison to the αCD3 scFv only (αCD3), i.e. without the PTD, shows very little activation, and correspondingly, the trimerized fusion protein of the invention (αCD3_{TAT}-Trimer) containing the HIV-Tat as the PTD has very significantly higher activation activity, in comparison to the trimerized αCD3 scFv only (αCD3), i.e. without the PTD. The activation is shown in FIG. 10A as proportion of CD4+ cells for CD25+ cells and for CD69+ cells, in FIG. 10B as proportion of CD4+ cells for IFNγ-positive cells, in FIG. 10C as proportion of CD8+ cells for CD25+ cells and for CD69+ cells, and in FIG. 10D as proportion of CD4+ cells for IFNγ-positive cells.

This demonstrates that the PTD domain of the fusion proteins of the invention very much contributes to the activation of splenocytes, whereas the αCD3 scFv alone has a rather small activity for activation of splenocytes.

As a further control, the fusion protein (αCD3-TAT), or its trimerized variant (αCD3-TAT-Trimer) was tested for activation of murine splenocytes in comparison to a protein without the αCD3-scFv (Hinge-TAT), corresponding to amino acids No. 1 . . . 21 directly linked to No. 279 . . . 530 of SEQ ID NO: 1, i.e. SEQ ID NO: 1 lacking amino acids No. 22 . . . 278. FIG. 11 shows the results, demonstrating that a protein without the CD3 binding scFv domain, i.e. the PTD domain and hinge, does not result in T cell-activation. FIG. 11A shows the proportion of CD4+ cells of CD25+ cells, showing that the background activation by the negative assay without added protein (control) was reached by the comparative Hinge-TAT, whereas the fusion protein of the invention (αCD3_{TAT}), as well as the trimerized fusion protein (αCD3_{TAT}-Trimer) resulted in effective activation. FIG. 11B shows the results for CD69+ cells, for CD8+ cells FIG. 11C shows the results for CD25+ cells, FIG. 11D for CD69+ cells.

FIG. 12A shows the proportion of CD4+ cells of IFNγ positive cells, FIG. 12B shows the proportion of CD8+ cells of IFNγ positive cells.

The results of FIG. 10 as well as the results of FIG. 11 and FIG. 12 confirm that only the fusion protein of the invention yields an effective activation of CD4+ cells and CD8+, whereas comparative scFv only or comparative PTD only yield very little or background activation only.

### EXAMPLE 7: USE OF FUSION PROTEIN OF THE INVENTION IN COMBINATION WITH αPD1 ANTIBODY IN TUMOUR TREATMENT

As depicted in FIG. 13A, a MC38 subcutaneous tumour was established in mice on day 0, 109 viral particles of the adenovirus serotype 5 (Ad5/11-p53) or viral particles expressing the fusion protein of the invention used in Example 3 were administered intra-tumour (i.t.) on day 5, followed by application of 75 μg anti-PD1 antibody (αPD1) on day 6 that was repeated twice a week on the days indicated. Log-rank (Mantel-Cox) test was used to calculate survival statistics. Significance of tumor volume measurements was assessed using two-tailed unpaired t-test *p≤0.05. Growth of individual tumors is shown on the right side (FIGS. 13B, 13C, and 13D).

FIG. 13B shows the growth of individual tumours on different mice in control treatment using 0.9% NaCl and αPD1 only, FIG. 13C shows individual tumour volumes of mice treated by empty viral particles (Ad5/11) as comparison in addition to αPD1, FIG. 13D shows tumour volumes of mice treated with viral particles expressing the fusion protein (Ad5/11-αCD3-TAT-Trimer) of the invention in combination with αPD1. Except for one of five animals, tumor growth was effectively suppressed by the exemplary oncolytic virus expressing the fusion protein of the invention in combination with subsequent administration of αPD1, whereas treatment with αPD1 only (NaCl 0.9% i.t.) shows that tumour growth can be delayed, and treatment αPD1 in combination with previous administration of the empty viral particles (Ad5/11) had only marginal positive effects against tumour growth.

FIG. 13E shows survival rates, FIG. 13F shows mean tumour volumes for tumour therapy using anti-PD1 antibody only (αPD1+0.9% NaCl) or anti-PD1 in combination with empty virus particles (αPD1+Ad5/11) for comparison and for the combination of anti-PD1 antibody with the fusion protein of the invention expressed from by an oncolytic virus particle (αPD1+Ad5/11-αCD3_{TAT}-Trimer).

The results show that the tumour growth and survival in αPD1 therapy of a solid tumour were significantly improved by the viral particles expressing the fusion protein of the invention. The anti-PD1 antibody, being representative of compounds blocking the PD-1/PD-L1 immune checkpoint, shows that the fusion protein, especially an oncolytic virus containing a nucleic acid construct encoding the fusion protein, is suitable for use in tumour therapy which comprises the administration of a compound blocking the PD-1/PD-L1 immune checkpoint.

FIG. 14 shows that expression of the fusion protein of the invention by oncolytic viruses during αPD1 checkpoint blockade improves the proximity of T cells and tumor cells as well as the interaction of CD4+ and CD8+ T cells.

Sub-cutaneous MC38 tumours established in mice were treated by a single intratumoral dose of Ad5/11-αCD3_{TAT}-Trimer, Ad5/11, and saline, respectively, each together with two applications of αPD1 antibodies as described above. Spatial phenotyping of intra-tumoral immune cells was assessed by multiplex immunohistochemical staining of CD8+ T cells (yellow), CD4+ T cells (green), CD161+NK cells (magenta), F4/80+ macrophages (white), pan-Cadherin+ (PC+) tumor cells (red) and nuclei (DAPI/blue). FIG. 14A, FIG. 14B, FIG. 14C show spatial immunohistochemical analysis of the proximity of T-cell subpopulations and tumor cells. Representative composite images from each group are shown in FIG. 14A, FIG. 14B and FIG. 14C. Cells within a maximum distance of 15 μm refer to a direct contact, close proximity is defined by a radius of maximum 30 μm, and near cells are found within a radius of 50 μm. The closest PC+ cell (tumour cell, identified as pan-Cadherin positive) to each CD8+ T cell is marked by a line. Quantitative analysis of the proximity between tumor cells and T cells is shown in FIG. 14D, FIG. 14E and FIG. 14F for CD8+ cells and FIG. 14G, FIG. 14H and FIG. 14I for CD4+ cells. These results show that in treatment in combination with αPD1-antibody, the fusion protein of the invention effective in localizing both CD8+ T-cells and CD4+ T-cells closer to tumour cells than αPD1-antibody alone or αPD1-antibody in combination with empty virus particles (Ad5/11).

FIG. 14J and FIG. 14K show the median distance of PC+ tumor cells to CD8+ T cells or CD4+ T cells. The spatial immunophenotyping analysis was used to investigate T-cell clustering within the tumor after treatment. Representative composite figures are shown in FIG. 14L, FIG. 14M and FIG. 14N. The distance analysis among CD8+ cells is shown in FIG. 14O, FIG. 14P and FIG. 14Q. FIG. 14R, FIG. 14S and FIG. 14T show the proximity analysis of CD8+ and CD4+ T cells. FIG. 14U and FIG. 14V show results of median distance measurements among CD8+ T cells, and between CD8+ and CD4+ T cells, respectively.

These data show that the intratumoral expression of a fusion protein of the invention, when expressed by an oncolytic virus in the tumour promote the clustering of CD4+ and CD8+ T cells relevant for mutual stimulation of these immune cell types.

1. A fusion protein that from N-terminus to C-terminus comprises or consists of an anti-CD3 antibody section, a hinge and a protein transduction domain (HIV-Tat domain).

2. The fusion protein according to claim 1, wherein the protein transduction domain is a peptide section of at least 5 amino acids up to 20 amino acids, e.g. up to 19 amino acids having at least 40% and/or at least 4 amino acids which are independently selected from the group of Arginine, Lysine and Histidine.

3. The fusion protein according to claim 1, wherein the fusion protein comprises an oligomerizing domain arranged between the hinge and the protein transduction domain.

4. The fusion protein according to claim 3, comprising a hydrophilic spacer combined with the oligomerizing domain, wherein the oligomerizing domain is a fibritin domain or a GCN4 domain.

5. The fusion-protein according to claim 4, wherein the fibritin domain has an amino acid sequence of at least 80% homology or identity to at least one of amino acids 521 . . . 550 of SEQ ID NO: 2, and amino acids 521 . . . 550 of SEQ ID NO: 4.

6. The fusion protein according to claim 1, wherein the hinge has an amino acid sequence of at least 80% homology or identity to at least one of amino acids 285 . . . 520 of SEQ ID NO: 1, amino acids 285 . . . 520 of SEQ ID NO: 2, amino acids 285 . . . 520 of SEQ ID NO: 3, and amino acids 285 . . . 520 of SEQ ID NO: 4.

7. The fusion protein according to claim 1, wherein the protein transduction domain has least 80% homology or identity to at least one of amino acids 521 . . . 530 of SEQ ID NO: 1, amino acids 557 . . . 566 of SEQ ID NO: 2, amino acids 521 . . . 530 of SEQ ID NO: 3, amino acids 557 . . . 566 of SEQ ID NO: 4, and SEQ ID NO: 20, or the PTD is SEQ ID NO: 81 or SEQ ID NO: 82.

8. The fusion protein according to claim 1, wherein the anti-CD3 antibody section is an anti-CD3 scFv specific for human CD3.

9. The fusion protein according to claim 1 for use in tumour treatment.

10. A nucleic acid construct encoding an anti-CD3 antibody section for use in tumour treatment, wherein the nucleic acid construct encodes a fusion protein according to claim 1.

11. The nucleic acid construct according to claim 10, contained in a viral particle.

12. The nucleic acid construct according to claim 10, contained in a liposome.

13. The nucleic acid construct according to claim 10, contained in an adenoviral particle.

14. The nucleic acid construct according to claim 10, wherein the nucleic acid construct comprises a nucleotide section of nucleotides 1 . . . 3706 and a nucleotide section of nucleotides 5986 . . . 37135 of SEQ ID NO: 13 and between these nucleotide sections comprises a coding sequence encoding the fusion protein under the control of a promoter, or comprises a nucleotide section of nucleotides 1 . . . 3706 and a nucleotide section of nucleotides 6094 . . . 37243 of SEQ ID NO: 71 and between these nucleotide sections comprises a coding sequence encoding the fusion protein under the control of a promoter.

15. The nucleic acid construct according to claim 10, wherein the coding sequence encoding the fusion protein is arranged under the control of a weak promoter.

16. The nucleic acid construct according to claim 10, wherein the coding sequence encoding the fusion protein is arranged in 3′ of an IRES, with a promoter arranged in 5′ of the IRES.

17. An oncolytic virus for use in the treatment of a solid tumour, the virus containing a nucleic acid construct according to claim 10.

18. The oncolytic virus according to claim 17, wherein the administration of the oncolytic virus is combined with the administration of a compound which blocks the PD-1/PD-L1 immune checkpoint.
